(19) United States
(12) Patent Application Publication
(10) Pub. No.: US 2020/0048305 A1
(43) Pub. Date: Feb. 13, 2020

(54) GLUTATHIONE-CHOLESTEROL DERIVATIVES AS BRAIN TARGETING AGENTS

(71) Applicant: South Dakota Board of Regents, Pierre, SD (US)

(72) Inventors: Xiangming Guan, Brookings, SD (US); Asidim Najmi, Brookings, SD (US); Shenggang Wang, Brookings, SD (US); Yue Huang, Brookings, SD (US); Teresa Seefeldt, Brookings, SD (US); Yahya Alqahtani, Brookings, SD (US)

(21) Appl. No.: 16/602,136

(22) Filed: Aug. 12, 2019

Related U.S. Application Data
(60) Provisional application No. 62/717,449, filed on Aug. 10, 2018.

(51) Int. Cl. C07K 5/02; A61K 47/28

(52) U.S. Cl. CPC C07K 5/0215; A61K 38/00; A61K 47/28

(57) ABSTRACT

The present invention describes compositions containing cholesterol-linker-glutathione conjugates for targeting the brain by overcoming barrier entry to the CNS through the blood brain barrier (BBB), including micelle and liposome forms of such compositions. In addition, methods for treating subjects by administering such compositions are also disclosed.

### CROSS REFERENCE TO RELATED APPLICATION

This application claims benefit under 35 U.S.C. § 119(e) to U.S. Provisional Application No. 62/717,449, filed Aug. 10, 2018, which is incorporated by reference herein in its entirety.

### BACKGROUND OF THE INVENTION

### Field of the Invention

Disclosed herein are methods and compositions increase delivery to the brain through blood brain barrier for treatment, prevention and/or diagnosis of CNS disorders or improving CNS conditions.

### Background Information

The blood-brain barrier (BBB) is a barrier that separates the peripheral blood circulation from the central nerve system (CNS). The BBB is characterized by a tight layer of endothelial cells that covers capillary blood vessels in the CNS. The barrier helps reduce or prevent xenobiotics from entering the CNS and is a protection mechanism for the CNS. However, the BBB also posts a barrier for therapeutics, diagnostics, and other xenobiotics to reach the CNS. Difficulty in reaching therapeutic concentration in the brain caused by the barrier has been the cause of treatment failure of brain diseases that include brain cancer, Alzheimer diseases, and Parkinson diseases.

Although the BBB blocks foreign compounds from entering the CNS, endogenous compounds, such as glucose, amino acids, peptides, neurotransmitters, and glutathione (GSH), enter the CNS readily through their corresponding receptors or transporters present in the BBB. Some of these receptors or transporters have been targeted to facilitate therapeutics, diagnostics, and other compounds to cross the BBB to reach the CNS.

GSH is an endogenous three amino acid peptide. It plays various roles in the body: as a major antioxidant, a compound that removes toxic compounds, and a compound involved in other cellular functions. GSH crosses the BBB through a Na-dependent GSH transporter. Recently, GSH transporters have been found effective in facilitating crossing of compounds through the BBB to reach the CNS. To achieve GSH transporter-mediated BBB crossing, GSH is linked to a therapeutic agent (GSH-Drug) to form a prodrug. The prodrug crosses the BBB by binding the GSH part to a GSH transporter followed by internalization of the prodrug.

GSH has also been linked to polyethylene glycol (PEG) which is connected to phospholipid (P) to form GSH-PEG-P or polyethylene glycol connected to vitamin E to form GSH-PEG-E. GSH-PEG-P and GSH-PEG-E have been coated on the surface of liposomes (GSH-PEGylated liposomes) to facilitate crossing of the liposomes through the BBB using the mechanism of binding the GSH moiety to a GSH transporter followed by internalization of the liposomes through endocytosis or transcytosis. The GSH-PEGylated liposomes have been shown to safely enhance the delivery to the brain by approximately 5-folds. However, these molecules are limited to liposome forms, which can be expensive to produce.

Accordingly, there is a need in the art for improved vehicles to deliver therapeutics across the BBB.

### SUMMARY OF THE INVENTION

The present invention discloses compositions that facilitate transport of various compounds through the blood brain barrier, which compositions contain cholesterol-glutathione based structures, including methods of treatment, prevention, and diagnosis of CNS disorders using such compositions.

In embodiments, a blood brain barrier permeable composition is disclosed, where the composition includes a molecule as set forth in Formula (I), (II), (III), (IV), (V), (VI), (VII) or (VIII):

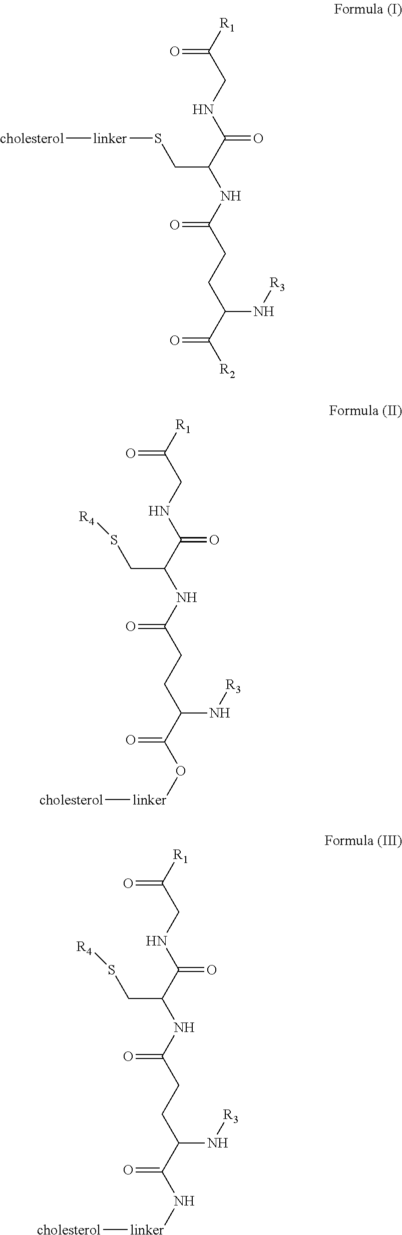

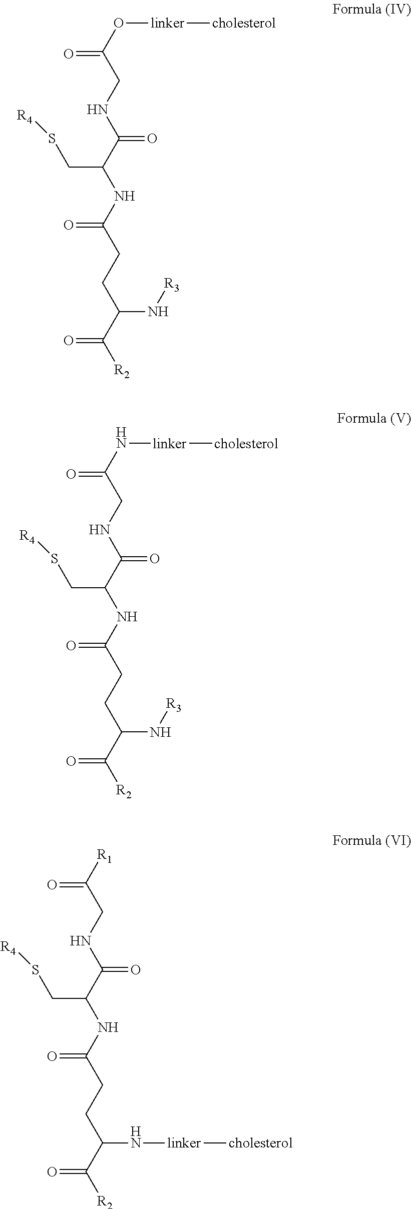

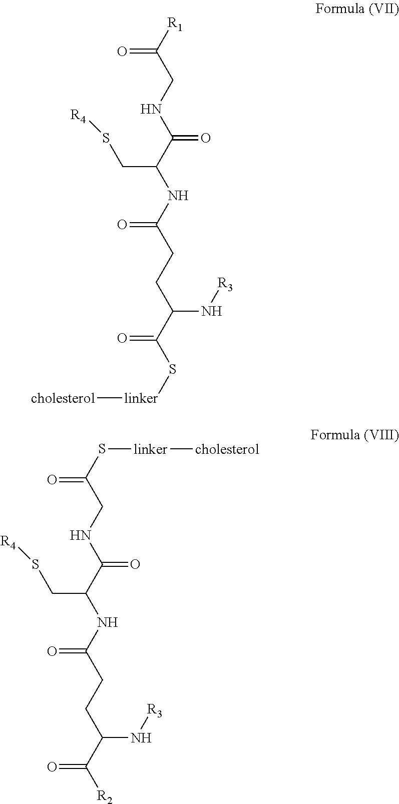

where R₁=OH, O-alkyl (1 to 30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), combinations thereof;
R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
R₃=H, alkyl (1-30 C), aryl (6-30 C), or aryl and alkyl combinations (6-30 C);
R₄=H, alkyl (1-30 C), aryl (6-30 C), or aryl and alkyl combinations (6-30 C), and
the linker includes alkyl, aliphatic chain, alkoxy and combinations thereof, where the composition does not fluoresce in the absence of an exogenous fluorescing compound.

In one aspect, the composition includes a molecule as set forth in Formula (I). In a related aspect, the composition comprises a molecule as set forth in Formula (IX):

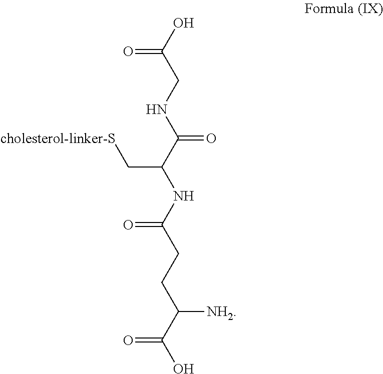

In one aspect, the linker includes at least one ethylene glycol moiety. In a related aspect, the linker includes two ethylene glycol moieties.

In one aspect, the composition exhibits a critical micellular concentration (CMC) of about 3.9 μM.

In embodiments, a blood brain barrier permeable composition is disclosed, where the composition includes a molecule as set forth in formula (I), (II), (III), (IV), (V), (VI), (VII) or (VIII):

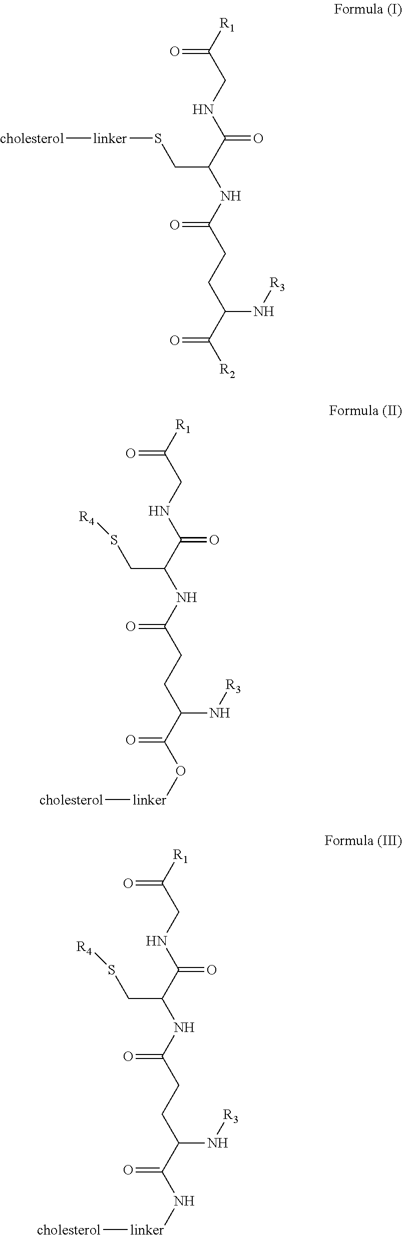

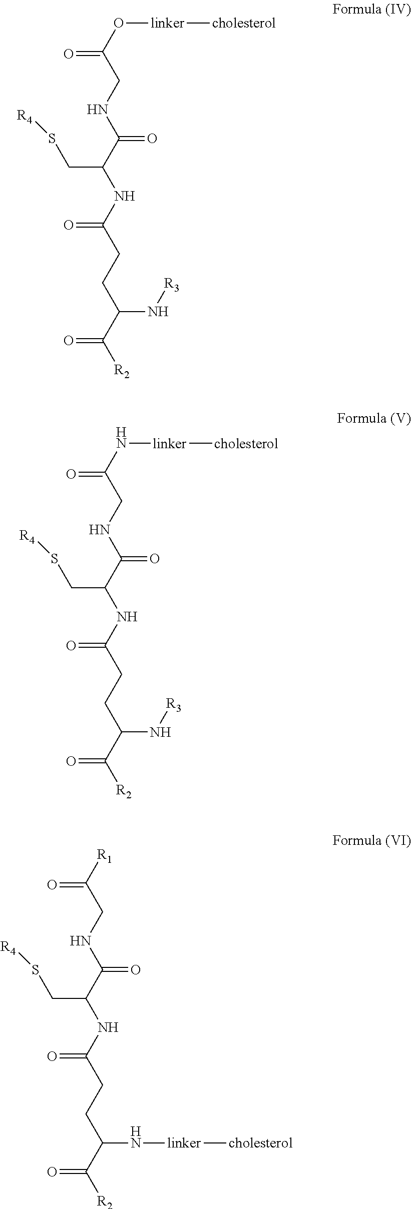

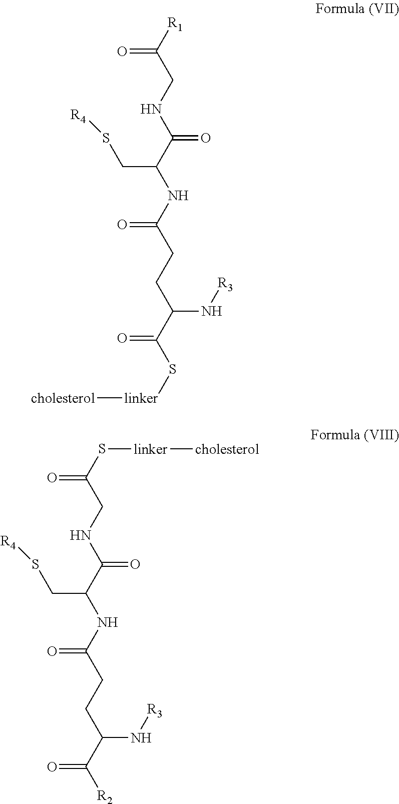

where R₁=R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
where R₃=R₄=H, alkyl (1-30 C), aryl (6-30 C), or aryl and alkyl combinations (6-30 C), and
the linker includes alkyl, aliphatic chain, alkoxy and combinations thereof, where the composition forms micelles.

In one aspect, the linker includes at least one ethylene glycol moiety. In a related aspect, the linker includes two ethylene glycol moieties.

In another aspect, the composition includes buffers, liposomes, dyes, fluorescent agents, drugs, chemotherapeutics, nutraceuticals, vitamins, nucleic acids, proteins, antigens, antibodies buffer, a hydrophobic agent, a therapeutic agent, an antioxidant, an anti-inflammatory drug, an anticancer drug, a cosmetic, a pharmaceutical, a pesticide, an insecticide, an herbicide, an antiseptic, a diagnostic agent, a food additive, a fragrance, a textile and combinations thereof.

In one aspect, further includes a hydrophobic agent. In a related aspect, the hydrophobic agent includes abietic acid, aceglatone, acenaphthene, acenocoumarol, acetohexamide, acetomeroctol, acetoxo lone, acetyldigitoxins, acetylene dibromide, acetylene dichloride, acetylsalicylic acid, alantolactone, aldrin, alexitol sodium, allethrin, allylestrenol, allyl sulfide, alprazolam, aluminum bis(acetylsalicylate), ambucetamide, aminochlothenoxazin, aminoglutethimide, amyl chloride, androstenediol, anethole trithone, anilazine, anthralin, Antimycin A, aplasmomycin, arsenoacetic acid, asiaticoside, astemizole, aurodox, aurothioglycanide, 8-azaguanine, azobenzene; baicalein, Balsam Peru, Balsam Tolu, barban, baxtrobin, bendazac, bendazol, bendroflumethiazide, benomyl, benzathine, benzestrol, benzodepa, benzoxiquinone, benzphetamine, benzthiazide, benzyl benzoate, benzyl cinnamate, bibrocathol, bifenox, binapacryl, bioresmethrin, bisabolol, bisacodyl, bis(chlorophenoxy)methane, bismuth iodosubgallate, bismuth subgallate, bismuth tannate, B isphenol A, bithionol, bornyl, bromoisovalerate, bornyl chloride, bornyl isovalerate, bornyl salicylate, brodifacoum, bromethalin, broxyquinoline, bufexamac, butamirate, butethal, buthiobate, butylated hydroxyanisole, butylated hydroxytoluene; calcium iodostearate, calcium saccharate, calcium stearate, capobenic acid, captan, carbamazepine, carbocloral, carbophenothin, carboquone, carotene, carvacrol, cephaeline, cephalin, chaulmoogric acid, chenodiol, chitin, chlordane, chlorfenac, chlorfenethol, chlorothalonil, chlorotrianisene, chlorprothixene, chlorquinaldol, chromonar, cilostazol, cinchonidine, citral, clinofibrate, clofazimine, clofibrate, cloflucarban, clonitrate, clopidol, clorindione, cloxazolam, coroxon, corticosterone, coumachlor, coumaphos, coumithoate cresyl acetate, crimidine, crufomate, cuprobam, cyamemazine, cyclandelate, cyclarbamate cymarin, cypernethril; dapsone, defosfamide, deltamethrin, deoxycorticocosterone acetate, desoximetasone, dextromoramide, diacetazoto, dialifor, diathymosulfone, decapthon, dichlofluani, dichlorophen, dichlorphenamide, dicofol, dicryl, dicumarol, dienestrol, diethylstilbestrol, difenamizole, dihydrocodeinone enol acetate, dihydroergotamine, dihydromorphine, dihydrotachysterol, dimestrol, dimethisterone, dioxathion, diphenane, N-(1,2-diphenylethyl)nicotinamide, dipyrocetyl, disulfamide, dithianone, doxenitoin, drazoxolon, durapatite, edifenphos, emodin, enfenamic acid, erbon, ergocominine, erythrityl tetranitrate, erythromycin stearate, estriol, ethaverine, ethisterone, ethyl biscoumacetate, ethylhydrocupreine, ethyl menthane carboxamide, eugenol, euprocin, exalamide; febarbamate, fenalamide, fenbendazole, fenipentol, fenitrothion, fenofibrate, fenquizone, fenthion, feprazone, flilpin, filixic acid, floctafenine, fluanisone, flumequine, fluocortin butyl, fluoxymesterone, fluorothyl, flutazolamn, fumagillin, 5-furfuryl-5-isopropylbarbituric acid, fusafungine, glafenine, glucagon, glutethimide, glybuthiazole, griseofulvin, guaiacol carbonate, guaiacol phosphate, halcinonide, hematoprophyrin, hexachlorophene, hexestrol, hexetidine, hexobarbital, hydrochlorothiazide, hydrocodone, ibuproxam, idebenone, indomethacin, inositol niacinate, iobenzamic acid, iocetamic acid, iodipamide, iomeglamic acid, ipodate, isometheptene, isonoxin, 2-isovalerylindane-1,3-dione, josamycin, 11-ketoprogesterone, laurocapram, 3-0-lauroylpyridoxol diacetate, lidocaine, lindane, linolenic acid, liothyronine, lucensomycin, mancozeb, mandelic acid, isoamyl ester, mazindol, mebendazole, mebhydroline, mebiquine, melarsoprol, melphalan, menadione, menthyl valerate, mephenoxalone, mephentermine, mephenyloin, meprylcaine, mestanolone, mestranol, mesulfen, metergoline, methallatal, methandriol, methaqualone, 3-methylcholanthrene, methylphenidate, 17-methyltestosterone, metipranolol, minaprine, myoral, naftalofos, naftopidil, naphthalene, 2-naphthyl lactate, 2-(2-naphthyloxy)ethanol, naphthyl salicylate, naproxen, nealbarbital, nemadectin, niclosamide, nicoclonate, nicomorphine, nifuroquine, nifuroxazide, nitracrine, nitromersol, nogalamycin, nordazepamn, norethandrolone, norgestrienone; octaverine, oleandrin, oleic acid, oxazepam, oxazolam, oxeladin, oxwthazaine, oxycodone, oxymesterone, oxyphenistan acetate, paclitaxel, paraherquamide, parathion, pemoline, pentaerythritol tetranitrate, pentylphenol, perphenazine, phencarb amide, pheniramine, 2-phenyl 6-chlorophenol, phentlmethylbarbituric acid, phenyloin, phosalone, phthalylsulfathiazole, phylloquinone, picadex, pifamine, piketopfen, piprozolin, pirozadil, plafibride, plaunotol, polaprezinc, polythiazide, probenecid, progesterone, promegestone, propanidid, propargite, propham, proquazone, protionamide, pyrimethamine, pyrimithate, pyrvinium pamoate, quercetin, quinbolone, quizalofo-ethyl, rafoxanide, rescinnamine, rociverine, runnel, salen, scarlet red, siccanin, simazine, simetride, sobuzoxane, solan, spironolactone, squalene, stanolone, sucralfate, sulfabenz, sulfaguanole, sulfasalazine, sulfoxide, sulpiride, suxibuzone, talbutal, tamoxifen, terguide, testosterone, tetrabromocresol, tetrandrine, thiacetazone, thiocolchicine, thioctic acid, thioquinox, thioridazine, thiram, thymyl N isoamylcarbamate, tioxidazole, tioxolone, tocopherol, tolciclate, tolnaftate, triclosan, triflusal, triparanol, ursolic acid, valinomycin, veraparnil, vinblastine, vitamin A, vitamin D, vitamin E, xenbucin, xylazine, zaltoprofen, zearalenone, and combinations thereof.

In one aspect, the composition is a micelle or a liposome.

In embodiments, a method of treating a disorder that affects the brain is disclosed including administering to a subject in need thereof a pharmaceutical composition comprising the blood brain barrier permeable composition at a sufficient concentration to relieve, prevent, and/or diagnose the symptoms of the disorder.

In one aspect, the disorder includes Alzheimer's disease, dementias, brain cancer, epilepsy, seizure disorders, mental disorders, Parkinson's disease, movement disorders, stroke and transient ischemic attacks.

In another aspect, the method further includes co-administration of glutathione.

In embodiments, a kit is disclosed including a blood brain barrier permeable composition, where the composition includes a molecule as set forth in: (i) formula (I), (II), (III), (IV), (V), (VI), (VII) or (VIII):

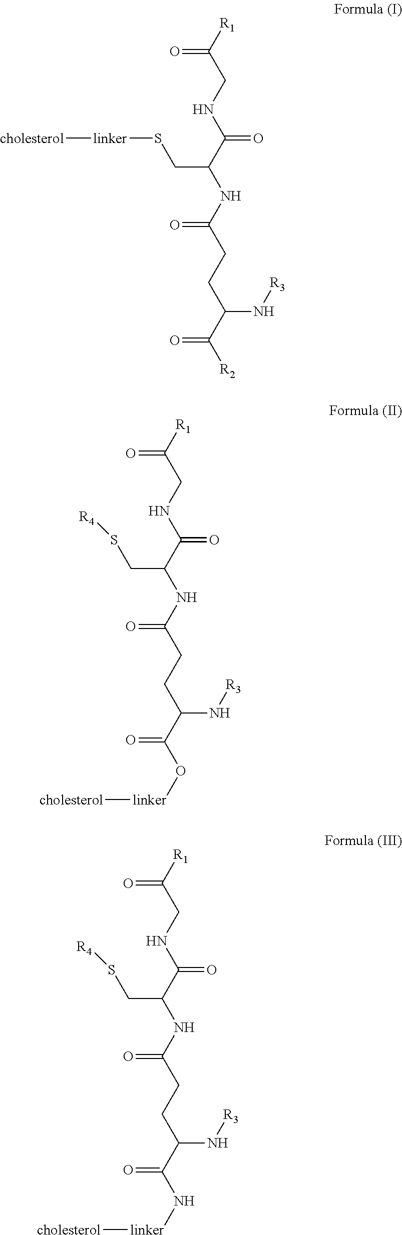

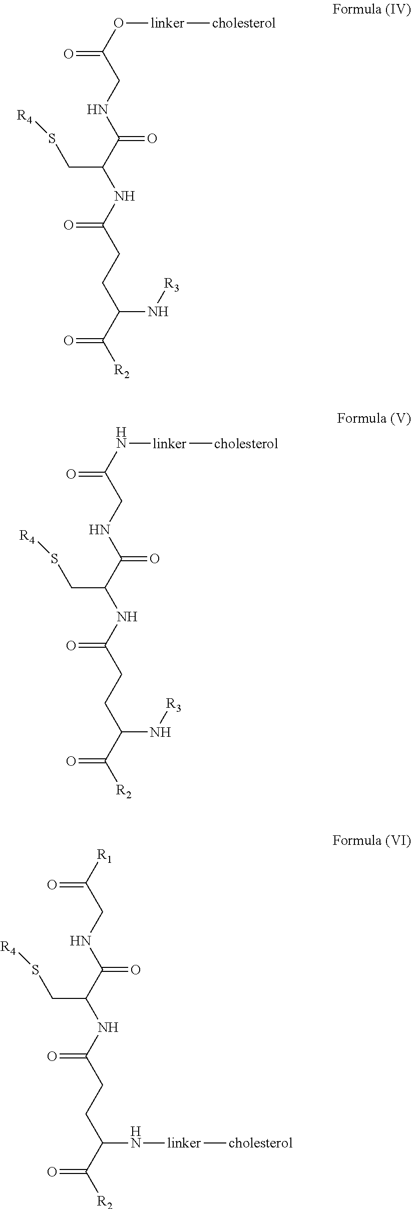

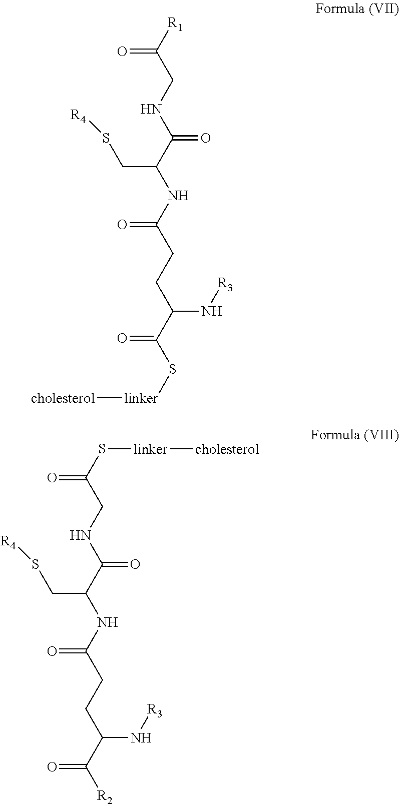

where R₁=OH, O-alkyl (1 to 30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), combinations thereof;
R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
R₃=H, alkyl (1-30 C), aryl (6-30 C), or aryl and alkyl combinations (6-30 C);
R₄=H, alkyl (1-30 C), aryl (6-30 C), or aryl and alkyl combinations (6-30 C), and
the linker includes alkyl, aliphatic chain, alkoxy and combinations thereof, where the composition does not fluoresce in the absence of an exogenous fluorescing compound or

(ii) a blood brain barrier permeable composition, where the composition includes a molecule as set forth in formula (I), (II), (III), (IV), (V), (VI), (VII) or (VIII):

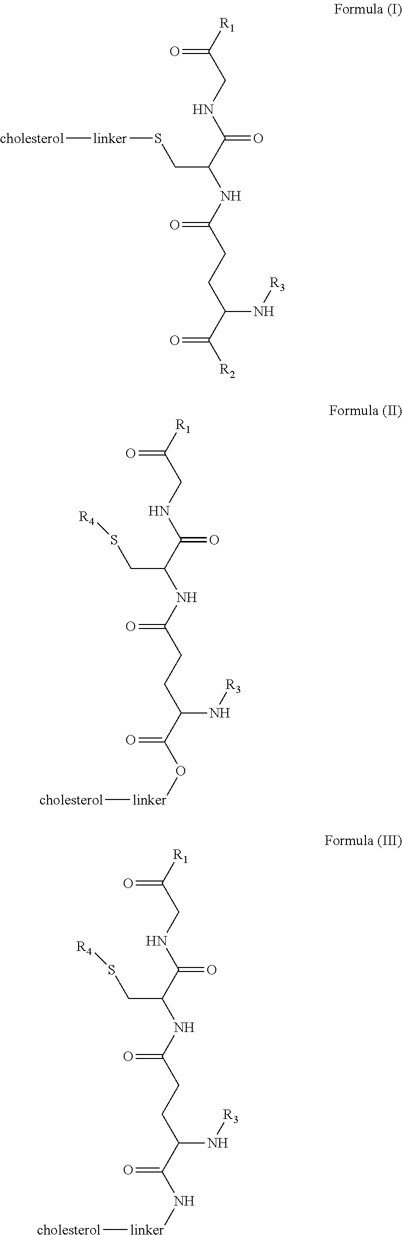

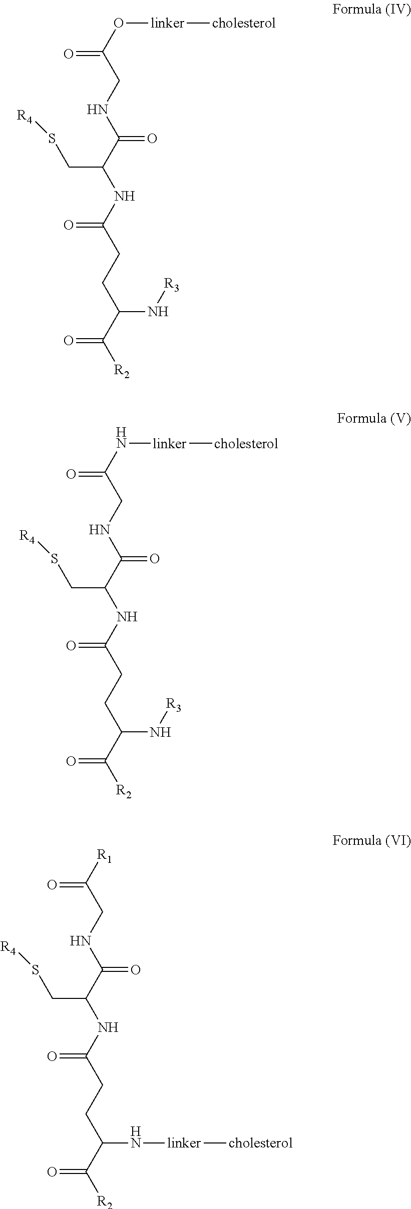

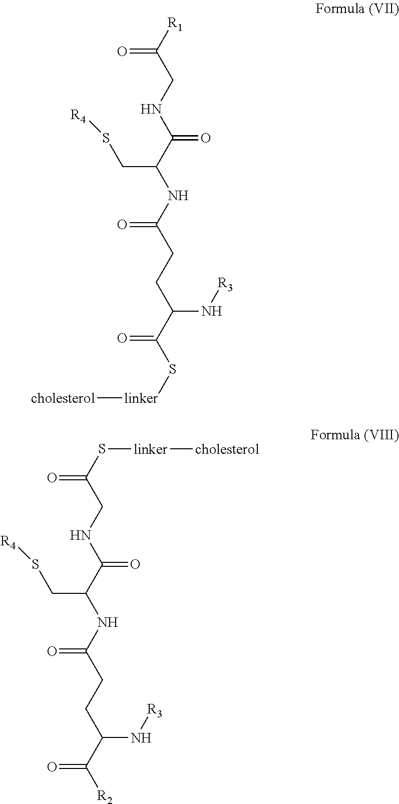

where R₁=R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
where R₃=R₄=H, alkyl (1-30 C), aryl (6-30 C), or aryl and alkyl combinations (6-30 C), and linker comprises alkyl, aliphatic chain, alkoxy and combinations thereof, where the composition forms micelles; b) optionally, one or more compounds selected from phospholipids, cholesterol or buffers; c) one or more agents including buffers, dyes, fluorescent agents, drugs, chemotherapeutics, nutraceuticals, vitamins, nucleic acids, proteins, antigens, antibodies buffer, a hydrophobic agent, a therapeutic agent, an antioxidant, an anti-inflammatory drug, an anticancer drug, a cosmetic, a pharmaceutical, a pesticide, an insecticide, an herbicide, an antiseptic, a diagnostic agent, a food additive, a fragrance, and a textile and combinations thereof; d) instructions reciting a method for producing a composition comprising a liposome or micelle and said one or more agents; e) a label; and f) a container including components (a)-(e).

In a related aspect, the blood brain barrier permeable composition comprises a molecule as set forth in Formula (IX):

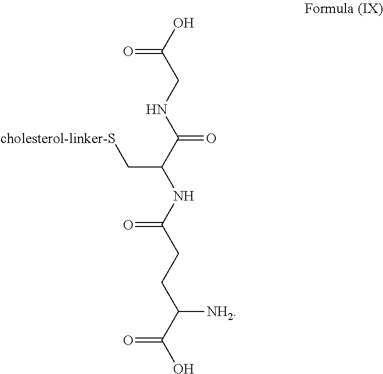

In embodiments, a medicament is disclosed including an effective amount of an intravenous composition containing the blood brain barrier permeable composition of for use in treating, preventing and/or diagnosing a disorder that affects the brain for the purpose of treating, preventing, diagnosing and/or relieving the symptoms of the disorder.

In a related aspect, the disorder includes Alzheimer's disease, dementias, brain cancer, epilepsy, seizure disorders, mental disorders, Parkinson's disease, movement disorders, stroke and transient ischemic attacks.

In a further related aspect, the composition further contains a separate amount of glutathione. In another aspect, the blood brain barrier permeable composition includes a molecule as set forth in Formula (IX):

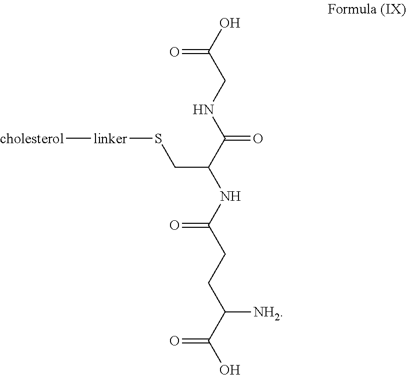

In one aspect, the medicament includes a liposome or a micelle.

### BRIEF DESCRIPTION OF THE DRAWINGS

FIG. 1 shows structures of the disclosed compositions, according to certain embodiments.

FIG. 2 shows the structure of the disclosed compounds according to certain embodiments.

FIG. 3 shows a representative HPLC chromatogram of GUNW-3 at concentration of 1 mg/ml (A) and the blank solvent (B).

FIG. 4 shows the chemical stability of GUNW-3 in a methanol-ammonium aqueous solution (1:1) at pH 10.8 and 4° C.

FIG. 5 shows the determination of the CMC of GUNW-3. Data are presented as the mean±SD (n=3).

FIG. 6 shows the stabilities of GUNW-3 DiR micelles and control DiR liposomes. Freshly prepared GUNW-3 DiR micelles and control DiR liposomes were checked, after a 0.5:100 dilution with deionized water, for particle size every 24 h at 4° C. for 5 days (mean±SD, n=3).

FIG. 7 shows the stabilities of GUNW-3 DiR micelles and control DiR liposomes in the presence of FBS. GUNW-3 DiR micelles (1 mg/mL) or control DiR liposome (1 mg/mL) were incubated in RPMI 1640 growth medium supplemented with 10% FBS at 37° C. Particle sizes were determined, after a 0.5:100 dilution with deionized water, on a Zetasizer (Malvern instrument, Westborough, Mass.).

FIG. 8 shows in-vivo whole body fluorescence imaging of mice at 15 min, 1 h, 24 h, and 48 h for free DiR (A), control DiR liposomes (B), and GUNW-3 DiR micelles (C). Lateral images for each group at 1 h were also presented. Mice were treated with GUNW-3 DiR liposomes (C, 0.2 mL containing 5 μg DiR, control DiR liposomes (B, 0.2 mL containing 5 μg DiR) and free DiR in ethanol (5 μg/25 μL) (C) by tail vein injection. The mice were under anesthesia using isoflurane and placed under a Bruker Xtream in-vivo imager for images taken at different time points. Pixel quantification of the region of interest was obtained for comparison.

FIG. 9 shows fold increase of GUNW-3 DiR micelles in the brain when compared with control DiR liposomes (C: B) and free DiR (C: A) based on the fluorescence intensity observed in the brain of the whole body imaging.

FIG. 10 shows ex-vivo imaging of the brains 1 h (10-I) or 48 h (10-II) after i.v. injection of free DiR (A), Control DiR liposomes (B), and GUNW-3 DiR micelles (C). Brains were harvested after heart perfusion with DPBS to remove blood in tissues. (10-ID): Semi-quantitative fluorescence intensity of the brains (C:A=12, C:B=5) (mean±SEM, n=3). (10-IID): Semi-quantitative fluorescence intensity of Brains (C:A=14, C:B=6.5) (mean±SEM, n=3).

FIG. 11 shows fold increase in brain Tamoxifen concentration achieved by GUNW-3 Tamoxifen micelles at 1 h and 24 h after dosing by tail vein injection when compared with dosing by free Tamoxifen in ethanol (5 μg/25 μL).

FIG. 12 shows stabilities of the GUNW-3 DiR liposomes and control DiR liposomes. Freshly prepared GUNW-3 DiR liposomes and control DiR liposomes were checked for particle sizes, after a 0.5:100 dilution with deionized water, every 24 h at 4° C. for 7 days on a Zetasizer (Malvern instrument, Westborough, Mass.) (mean±SD, n=3).

FIG. 13 shows stabilities of GUNW-3 DiR liposomes and control DiR liposomes in the presence of FBS. GUNW-3 DiR liposomes (1 mg/mL) or control DiR liposomes (1 mg/mL) were incubated in RPMI 1640 growth medium supplemented with 10% FBS at 37° C. After a 0.5:100 dilution with deionized water, particle sizes were checked on a Zetasizer (Malvern instrument, Westborough, Mass.) (mean±SD, n=3).

FIG. 14 shows in-vivo whole body fluorescence imaging of mice treated with control DiR liposomes (A) or GUNW-3 DiR liposomes (B). Females BALB/Cj mice (6-8 weeks old, 17-20 g) from Jackson Laboratory (Bar Harbor, Me., USA) and were acclimatized to laboratory condition for one week before the experiment. Mice were divided into two groups and intravenously injected with GUNW-3 DiR liposomes or control DiR liposome (250 μg DiR/kg) through the tail vain. Optical images of the whole body were taken at 5 min, 15 min, 30 min, 60 min, and 180 min. Images were analyzed and measured using Bruker MI SE software.

FIG. 15 shows a comparison of the fluorescence intensity of the mouse brains presented in FIG. 14.

FIG. 16 shows ex-vivo imaging of the brains collected 1 h after i.v. injection of control DiR liposomes (A) or GUNW-3 DiR liposomes (B). Blood was removed through heart perfusion before tissues were collected. The results were presented as fluorescence intensity detected from the isolated brain (mean±SEM, n=3).

FIG. 17 shows stabilities of the GUNW-3 Tamoxifen liposomes and control Tamoxifen liposomes. Freshly prepared GUNW-3 Tamoxifen liposomes and control Tamoxifen liposomes were checked for particle sizes, after a 0.5:100 dilution with deionized water, every 24 h at 4° C. for 5 days on a Zetasizer (Malvern instrument, Westborough, Mass.) (mean±SD).

FIG. 18 shows tamoxifen brain accumulation in mice treated by GUNW-3 Tamoxifen liposomes or control liposome 1 h after an IV injection through the tail vein (mean±SEM). Females BALB/Cj mice, [6-8 weeks old, 17-20 g from Jackson Laboratory (Bar Harbor, Me., USA)] were acclimatized to the laboratory condition for one week before the experiment. Mice were divided into two groups and intravenously injected with GUNW-3 Tamoxifen liposomes (10 mg/kg tamoxifen) or control Tamoxifen liposomes (10 mg tamoxifen/Kg) through the tail vain. Mice were sacrificed 1 h post dosage, and heart were perfused before tissues were collected. Tissues were kept at −80° C. for future analysis. The concentration of Tamoxifen in brains was determined by LC/MS/MS.

FIG. 19 shows brain-targeting of GUNW-3 DiR micelles with co-injection of GSH. I: In-vivo whole body fluorescence imaging of mice treated with GUNW-3 DiR micelles (A) and GUNW-3 DiR micelles plus GSH (B). Mice were treated with GUNW-3 DiR micelles (A, 0.2 mL containing 5 μg DiR), GUNW-3 DiR micelles+GSH (B, 0.2 mL containing 5 μg DiR and 1.98 mg GSH) by tail vein injection. The mice were under anesthesia using isoflurane and placed under a Bruker Xtream in-vivo imager for images taken at 1 h. Pixel quantification of the region of interest was obtained for comparison. II: Ex-vivo imaging of the brains collected 1 h after i.v. injection of GUNW-3 DiR micelles (A) or GUNW-3 DiR micelles plus GSH (B). Blood was removed through heart perfusion before tissues were collected. The results were presented as fluorescence intensity detected from the isolated brains (mean±SEM, n=3). III: A bar graph based on the fluorescence intensity from the corresponding brains in FIG. 19-II.

FIG. 20 shows two vials with the first vial (cloudy) containing Taxol (Paclitaxel) dissolved in Cremophor EL-ethanol (1:1) and diluted in 0.9% (w/v) sodium chloride as reported for an IV solution of Taxol6 and the other vial with same amount of Taxol (1 mg/mL) dissolved in aqueous GUNW-2 micelles.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a molecule” includes one or more molecules, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus<10% of particular term and “substantially” and “significantly” will mean plus or minus>10% of the particular term. In embodiments, composition may “contain”, “comprise” or “consist essentially of” a particular component or group of components, where the skilled artisan would understand the latter to mean the scope of the claim is limited to the specified materials or steps “and those that do not materially affect the basic and novel characteristic(s)” of the claimed invention.

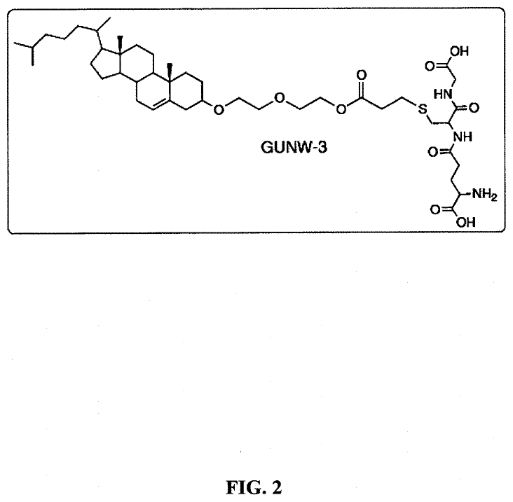

In embodiments, cholesterol-GSH containing molecules are disclosed as brain targeting agents. One of the disclosed molecules is referred to herein as GUNW-3, and is best shown in FIG. 2.

Further, as disclosed herein, experiments in mice show that GUNW-3 improves brain delivery of liposomes by 21 fold. In contrast, GSH-PEG-P, a composition developed for CNS drug delivery, which has been tested in clinical trials, exhibited 5 fold increase in brain delivery. As shown herein, brain delivery of micelles made of GUNW-3 was even more brain selective than liposomes.

In one aspect, GUNW-3 was designed by connecting a hydrophilic GSH molecule to a hydrophobic cholesterol molecule through a two ethylene glycol-unit linker. While not being bound by theory, the GSH moiety serves as a brain-targeting structure through binding to the GSH transporter, thus, facilitating the entry of the vehicle into the brain. Accordingly, the agents may be used to deliver compounds (e.g., but not limited to, therapeutics, diagnostics and preventative agents and the like) to reach the brain through the following manners:

1. The molecules were found to have the capacity to form micelles, having a CMC value of about 3.9 μM. As shown herein, data confirm that GUNW-3 micelles were able to significantly (>3 folds) improve the delivery of compounds to the brain and retain in the brain well by a single injection.

2. The molecules were able to embed the hydrophobic cholesterol moiety into the liposome double lipid layer with the hydrophilic GSH moiety floating on the surface of the liposomes for brain targeting to direct the liposomes to the brain. As disclosed herein, data confirms that GUNW-3 liposomes were able to significantly (>3 folds) improve the delivery of compounds to the brain and be retained in the brain well by a single injection.

Liposomes and micelles are known effective drug carriers that may be used to deliver various drugs or compounds that include small molecule therapeutics, DNA, RNA, and proteins (e.g., antibodies). Liposomes and micelles may encapsulate drugs and protect them from in vivo/in vitro degradation. They may also help reduce drug clearance, increase in vivo drug half-life, enhance the drug payload, control drug release, and improve drug-solubility.

Therefore, the abilities of cholesterol-GSH containing molecules to form brain-targeting micelles and brain-targeting liposomes find a broad application in therapeutic, diagnostic, and preventive treatments of various CNS diseases.

3. The molecules may be linked to a drug molecule through a metabolically cleavable bond to form a prodrug and transport the prodrug to the brain, then release the drug in the brain.

In addition to the above,

i) as shown herein, tests with mice reveal that co-injection of GSH with GUNW-3 micelles may further increase brain-targeting effect by ˜2 folds. In other word, co-injection of GSH with GUNW-3 micelles can increase brain-targeting by a total of ≥6 folds. While not being bound by theory, co-injection of GSH with GUNW-3 liposomes would be expected have the same effects;

ii) as shown herein, GUNW-3 can substantially increase the water solubility of taxol and thus make taxol an IV injectable solution. This would have a great application in taxol therapeutic treatment since poor water solubility of taxol remains challenging. In a related aspect, GUNW-3 may also increase aqueous solubility for other water insoluble compounds. For example such drugs include, but are not limited to, abietic acid, aceglatone, acenaphthene, acenocoumarol, acetohexamide, acetomeroctol, acetoxolone, acetyldigitoxins, acetylene dibromide, acetylene dichloride, acetylsalicylic acid, alantolactone, aldrin, alexitol sodium, allethrin, allylestrenol, allyl sulfide, alprazolam, aluminum bis(acetylsalicylate), ambucetamide, aminochlothenoxazin, aminoglutethimide, amyl chloride, androstenediol, anethole trithone, anilazine, anthralin, Antimycin A, aplasmomycin, arsenoacetic acid, asiaticoside, astemizole, aurodox, aurothioglycanide, 8-azaguanine, azobenzene; baicalein, Balsam Peru, Balsam Tolu, barban, baxtrobin, bendazac, bendazol, bendroflumethiazide, benomyl, benzathine, benzestrol, benzodepa, benzoxiquinone, benzphetamine, benzthiazide, benzyl benzoate, benzyl cinnamate, bibrocathol, bifenox, binapacryl, bioresmethrin, bisabolol, bisacodyl, bis(chlorophenoxy)methane, bismuth iodosubgallate, bismuth subgallate, bismuth tannate, Bisphenol A, bithionol, bornyl, bromoisovalerate, bornyl chloride, bornyl isovalerate, bornyl salicylate, brodifacoum, bromethalin, broxyquinoline, bufexamac, butamirate, butethal, buthiobate, butylated hydroxyanisole, butylated hydroxytoluene; calcium iodostearate, calcium saccharate, calcium stearate, capobenic acid, captan, carbamazepine, carbocloral, carbophenothin, carboquone, carotene, carvacrol, cephaeline, cephalin, chaulmoogric acid, chenodiol, chitin, chlordane, chlorfenac, chlorfenethol, chlorothalonil, chlorotrianisene, chlorprothixene, chlorquinaldol, chromonar, cilostazol, cinchonidine, citral, clinofibrate, clofazimine, clofibrate, cloflucarban, clonitrate, clopidol, clorindione, cloxazolam, coroxon, corticosterone, coumachlor, coumaphos, coumithoate cresyl acetate, crimidine, crufomate, cuprobam, cyamemazine, cyclandelate, cyclarbamate cymarin, cypernethril; dapsone, defosfamide, deltamethrin, deoxycorticocosterone acetate, desoximetasone, dextromoramide, diacetazoto, dialifor, diathymosulfone, decapthon, dichlofluani, dichlorophen, dichlorphenamide, dicofol, dicryl, dicumarol, dienestrol, diethylstilbestrol, difenamizole, dihydrocodeinone enol acetate, dihydroergotamine, dihydromorphine, dihydrotachysterol, dimestrol, dimethisterone, dioxathion, diphenane, N-(1,2-diphenylethyl)nicotinamide, dipyrocetyl, disulfamide, dithianone, doxenitoin, drazoxolon, durapatite, edifenphos, emodin, enfenamic acid, erbon, ergocominine, erythrityl tetranitrate, erythromycin stearate, estriol, ethaverine, ethisterone, ethyl biscoumacetate, ethylhydrocupreine, ethyl menthane carboxamide, eugenol, euprocin, exalamide; febarbamate, fenalamide, fenbendazole, fenipentol, fenitrothion, fenofibrate, fenquizone, fenthion, feprazone, flilpin, filixic acid, floctafenine, fluanisone, flumequine, fluocortin butyl, fluoxymesterone, fluorothyl, flutazolamn, fumagillin, 5-furfuryl-5-isopropylbarbituric acid, fusafungine, glafenine, glucagon, glutethimide, glybuthiazole, griseofulvin, guaiacol carbonate, guaiacol phosphate, halcinonide, hematoprophyrin, hexachlorophene, hexestrol, hexetidine, hexobarbital, hydrochlorothiazide, hydrocodone, ibuproxam, idebenone, indomethacin, inositol niacinate, iobenzamic acid, iocetamic acid, iodipamide, iomeglamic acid, ipodate, isometheptene, isonoxin, 2-isovalerylindane-1,3-dione, josamycin, 11-ketoprogesterone, laurocapram, 3-0-lauroylpyridoxol diacetate, lidocaine, lindane, linolenic acid, liothyronine, lucensomycin, mancozeb, mandelic acid, isoamyl ester, mazindol, mebendazole, mebhydroline, mebiquine, melarsoprol, melphalan, menadione, menthyl valerate, mephenoxalone, mephentermine, mephenyloin, meprylcaine, mestanolone, mestranol, mesulfen, metergoline, methallatal, methandriol, methaqualone, 3-methylcholanthrene, methylphenidate, 17-methyltestosterone, metipranolol, minaprine, myoral, naftalofos, naftopidil, naphthalene, 2-naphthyl lactate, 2-(2-naphthyloxy)ethanol, naphthyl salicylate, naproxen, nealbarbital, nemadectin, niclosamide, nicoclonate, nicomorphine, nifuroquine, nifuroxazide, nitracrine, nitromersol, nogalamycin, nordazepamn, norethandrolone, norgestrienone; octaverine, oleandrin, oleic acid, oxazepam, oxazolam, oxeladin, oxwthazaine, oxycodone, oxymesterone, oxyphenistan acetate, paclitaxel, paraherquamide, parathion, pemoline, pentaerythritol tetranitrate, pentylphenol, perphenazine, phencarb amide, pheniramine, 2-phenyl 6-chlorophenol, phentlmethylbarbituric acid, phenyloin, phosalone, phthalylsulfathiazole, phylloquinone, picadex, pifamine, piketopfen, piprozolin, pirozadil, plafibride, plaunotol, polaprezinc, polythiazide, probenecid, progesterone, promegestone, propanidid, propargite, propham, proquazone, protionamide, pyrimethamine, pyrimithate, pyrvinium pamoate, quercetin, quinbolone, quizalofo-ethyl, rafoxanide, rescinnamine, rociverine, runnel, salen, scarlet red, siccanin, simazine, simetride, sobuzoxane, solan, spironolactone, squalene, stanolone, sucralfate, sulfabenz, sulfaguanole, sulfasalazine, sulfoxide, sulpiride, suxibuzone, talbutal, tamoxifen, terguide, testosterone, tetrabromocresol, tetrandrine, thiacetazone, thiocolchicine, thioctic acid, thioquinox, thioridazine, thiram, thymyl N isoamylcarbamate, tioxidazole, tioxolone, tocopherol, tolciclate, tolnaftate, triclosan, triflusal, triparanol, ursolic acid, valinomycin, veraparnil, vinblastine, vitamin A, vitamin D, vitamin E, xenbucin, xylazine, zaltoprofen, zearalenone, and combinations thereof.

As disclosed herein, GUNW-3 was designed by connecting a hydrophilic GSH molecule to a hydrophobic cholesterol molecule through a two ethylene glycol unit linker (FIG. 2), although other linkers may be employed as would be apparent to one of skill in the art. While not being bound by theory, the GSH moiety serves as a brain-targeting structure through binding to a GSH transporter which facilitates the entry of the molecule into the brain. The GSH moiety is also hydrophilic or water soluble, which increases water solubility of the molecule. The cholesterol moiety is hydrophobic. While not being bound by theory, since cholesterol may embed into liposome structures, the cholesterol moiety of GUNW-3 is expected, like a cholesterol molecule, to be embedded into the double lipid layer of liposomes. Again, while not being bound by theory, the short chain linker is provided to hold the brain targeting part (GSH) close to the part to be delivered (such as liposomes or micelles) to make the brain-targeting effective vs. a long chain (such as PEG with a molecule weight>1000), where the latter separates the brain targeting moiety at quite a distance from the part to be delivered, and may in fact reduce the brain targeting effect. In a related aspect, bulky groups within the linker may sterically hinder micelle formation.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “conjugate”, including grammatical variations thereof, means a compound formed by the joining of two or more chemical compounds.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

“R₁,” “R₂,” “R₃,” “Rₙ,” where n is an integer, as used herein can, independently, possess one or more of the groups listed above. For example, if R₁ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, and the like, of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “pharmaceutically acceptable carrier” or “carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers. In a related aspect, the particle size is less than or equal to 150 nm.

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more CNS disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount/concentration”, “sufficient amount/concentration” and “amount effective” refer to an amount that is ample enough to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Disclosed herein is a composition for increasing penetration of the BBB comprising a cholesterol moiety; a glutathione (GSH) moiety; and a linker connecting the cholesterol moiety and the GSH moiety and one or more CNS drug compound or a compound that can benefit the CNS condition such as improving CNS functions or preventing CNS dysfunctions conjugated thereto.

According to certain embodiments, the composition further comprises a pharmaceutically acceptable nanocarrier. In certain embodiments, the pharmaceutically acceptable nanocarrier is a micelle. In further embodiments, the pharmaceutically acceptable nanocarrier is a liposome.

Numerous endogenous compounds, such as glucose, amino acids, peptides, neurotransmitters, and glutathione (GSH), enter the CNS through their corresponding receptors or transporters present in the BBB. Some of these receptors or transporters have been employed to facilitate therapeutics, diagnostics, and other compounds to cross the BBB to reach the CNS.

GSH is an endogenous three amino acid peptide. GSH plays various roles in the body: as a major antioxidant, a compound that removes reactive electrophiles (toxic compounds), and a compound involved in other cellular functions. GSH crosses the BBB through Na-dependent GSH transporters. GSH transporters are effective in facilitating crossing of compounds through the BBB to reach the CNS. To achieve GSH transporter-facilitated BBB crossing, GSH has been linked to a therapeutic agent (GSH-Drug) to form a prodrug. The prodrug crosses the BBB by binding the moiety of GSH to a GSH transporter followed by internalization of the prodrug. GSH has also been linked to polyethylene glycol (PEG) which is connected to phospholipid (P) to form GSH¬PEG-P or polyethylene glycol connected to vitamin E to form GSH-PEG-E. GSH-PEG-P and GSH-PEG-E can serve as brain-targeting. GSH-PEG-P and GSH-PEG-E have been coated on the surface of liposomes (GSH-PEGylated liposomes) to facilitate crossing of the liposomes through the BBB using the mechanism of binding the GSH moiety to a GSH transporter followed by internalization of the liposomes by endocytosis or transcytosis. The GSH-PEGylated liposomes have been shown to safely enhance the delivery to the brain by approximately 5-folds.

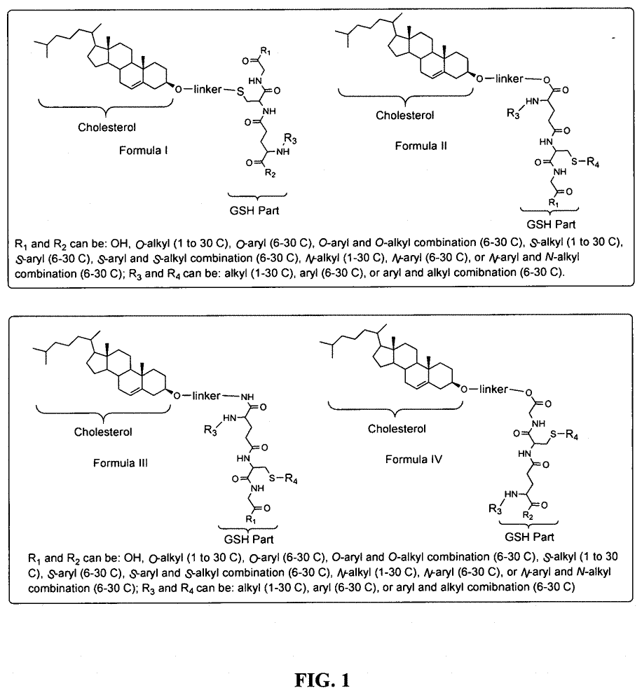

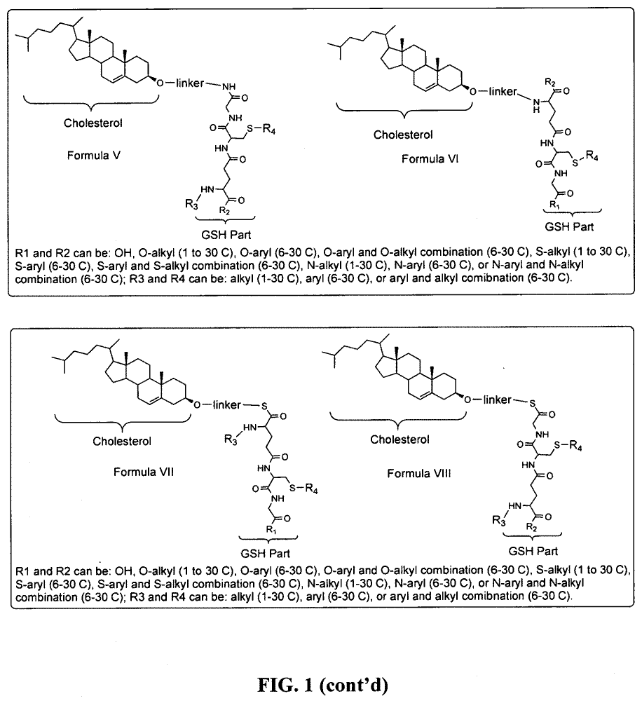

The instantly disclosed compounds are molecules that can help deliver compounds (therapeutics, diagnostic, and other compounds) to the brain with high efficiency through GSH transporters. Specifically, GSH based brain targeting molecules are disclosed. According to certain embodiments, as shown in FIG. 1, the instantly disclosed designed brain-targeting molecules have a common structural feature that is a GSH moiety linked to a cholesterol moiety through a linker. These designed molecules have a hydrophilic head (GSH) and a hydrophobic tail (cholesterol). This structural feature is capable of forming micelles with the hydrophilic head (GSH) being on the surface of the micelles. Since cholesterol is a composition of liposomes, the designed molecules may also be used for preparation of liposomes in which the hydrophobic tail (cholesterol) will be imbedded into the hydrophobic layer of liposomes while the hydrophilic head (GSH) will be on the surface of the liposomes. In both micelle and liposomes cases, GSH on the surface will bind to GSH transporters and enable the micelles or liposomes to cross the BBB to reach the CNS. Micelles and liposomes are known drug delivery systems that can carry compounds (therapeutics, diagnostic, or other compounds). Therefore, micelles and liposomes comprising the instantly disclosed compounds function as delivery systems for therapeutics, diagnostics, or other compounds with an enhanced ability to reach the brain.

According to certain embodiments, the linker is a hydrocarbon chain with a molecular weight of 0 to 10,000. In certain aspects, the linker may contain heteroatoms. According to certain embodiments, the composition of the linker improves the brain targeting efficiency and/or other properties such as pharmaceutical and pharmacokinetic properties.

According to certain embodiments, the cholesterol moiety may comprise structural modification on the cholesterol structure such as reduction of the double bond or addition of hydrocarbon (1-30 carbons) or other functional groups such as —OH, —NH₂, COOH at various positions to improve the brain targeting efficiency and/or other properties such as pharmaceutical and pharmacokinetic properties.

These agents may be used to form micelles, liposomes, and other nanocarriers to deliver therapeutics, diagnostics, and other compounds to the CNS to treat or to prevent brain diseases, or used for diagnostic purpose for brain diseases. They may also be used to aid the delivery of therapeutics, diagnostics, and other compounds to cells, tissues/organs with GSH transporters. In addition, these agents may be linked directly to a therapeutic, diagnostic, and other compounds for brain delivery or delivery to cells, tissues/organs with GSH transporters.

In contrast to previous brain delivery compositions such as GSH-PEG-P and GSH-PEG-E, the instantly disclosed compositions has the feature of GSH-linker-cholesterol. Cholesterol is different than P (phospholipid) and E (vitamin E). GUNW-3 has two ethylene-1,2-diol units with a molecular weight of 106, which is substantially different than PEG used in previous compositions, which has a molecular weight of 2000. Furthermore, the instantly claim composition achieved substantially greater or unexpected brain targeting selectivity: 21 folds for ours vs. 5 folds for past compositions. GUNW-3, also yields low distribution in liver when delivered in micelles—which is contrary to what would be expected from micelle and liposome delivery.

In certain aspects, the composition is administered in a therapeutically effective amount. In further aspects, the composition is administered in a prophylactically effective amount.

In further aspects, the composition administered to the subject may be in a range of about 0.001 mg/kg to about 1000 mg/kg.

In certain aspects, the method further comprises administering the composition as a bolus and/or at regular intervals. In certain aspects, the disclosed method further comprises administering the composition intravenously, intraperitoneally, intramuscularly, orally, subcutaneously, or transdermally.

In certain aspects, the compositions may be used in assays to diagnose diseases including, but not limited to, CNS disorders.

The following examples are intended to illustrate but not limit the invention.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1. Synthesis of GUNW-3

According to certain embodiments, GUNW-3 is synthesized according to the following method:

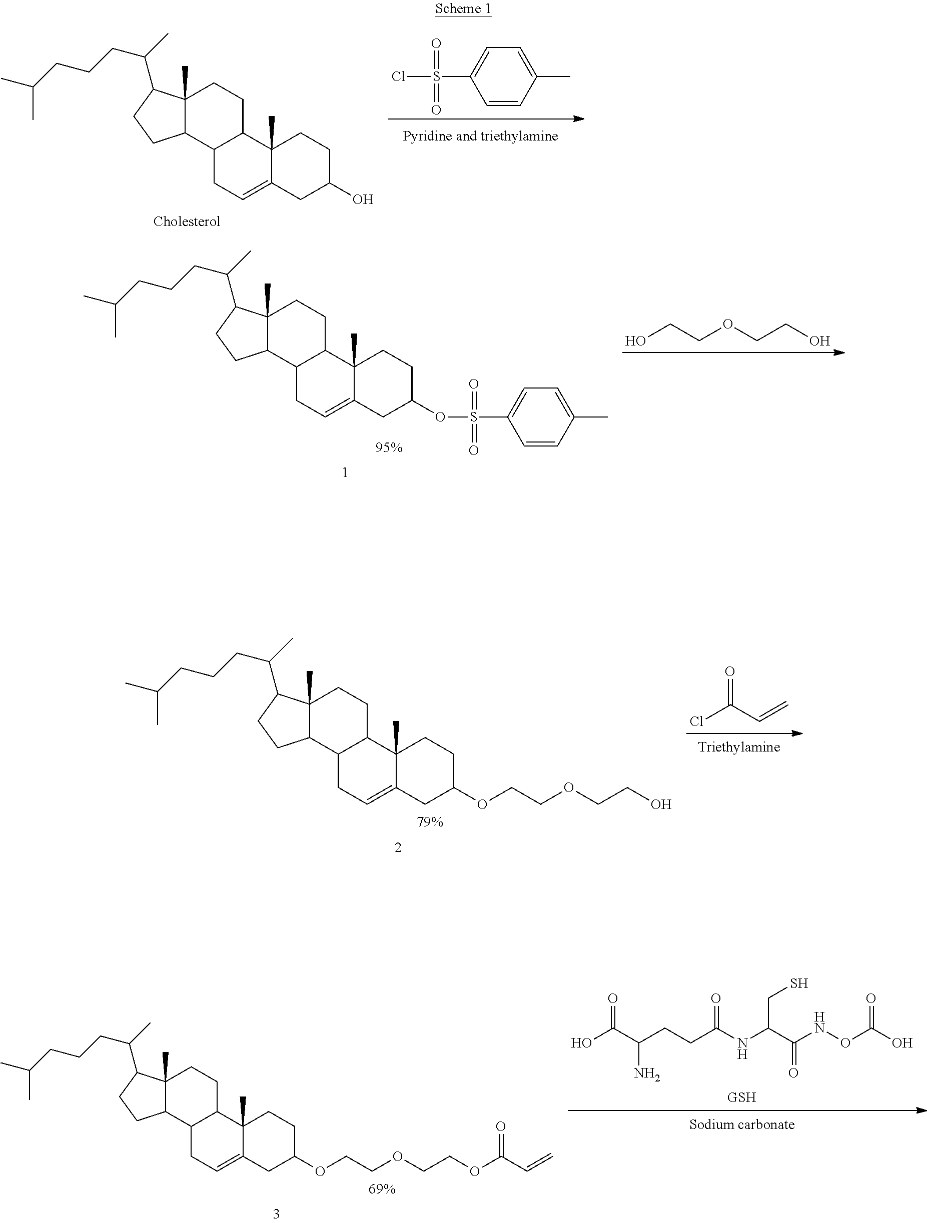

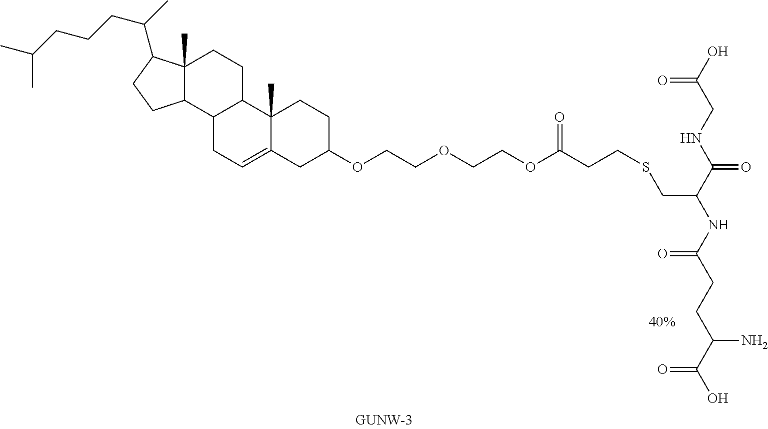 [SMILES: CC(C)CCCC(C)C1CCC2C3CC=C4CC(OCCOCCOC(=O)CCSCC(NC(=O)CCC(N)C(=O)O)C(=O)NCC(=O)O)CC[C@@]4(C)C3CC[C@@]12C]

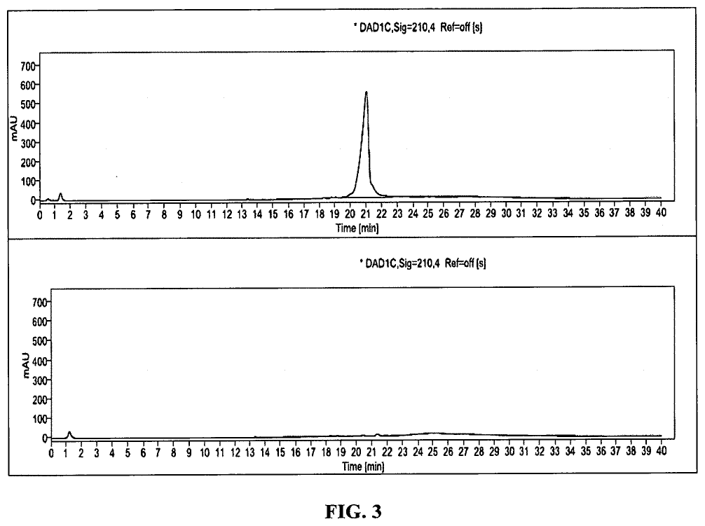

Briefly, GUNW-3 was synthesized in a total of 4 steps as outlined in Scheme I. The first step was tosylation of a commercially available cholesterol with 4-methylbenzene-1-sulfonyl chloride in the presence of pyridine and triethylamine to produce tosylated cholesterol (1) with 95% yield. Compound 1 was added with ethylene glycol to produce cholesterol-ethylene glycol (2) with 79% yield. Cholesterol-ethylene glycol (2) reacted with acryloyl chloride in the presence of triethylamine to form cholesterol-ethyleneglycol-acrylate (3) with 69% yield. Michael addition coupling of GSH to compound 3 was achieved in the presence of sodium carbonate to complete the synthesis of GUNW-3, with 40% yield. GUNW-3 was characterized by ¹H NMR and HRMS. The purity of GUNW-3 was confirmed to be 97% by HPLC (FIG. 3).

i. Purity and Stability of GUNW-3

The purity of GUNW-3 was checked by HPLC and determined to be 97% as shown by a representative HPLC chromatogram in FIG. 3A. FIG. 3B is a representative HPLC chromatogram from solvents.

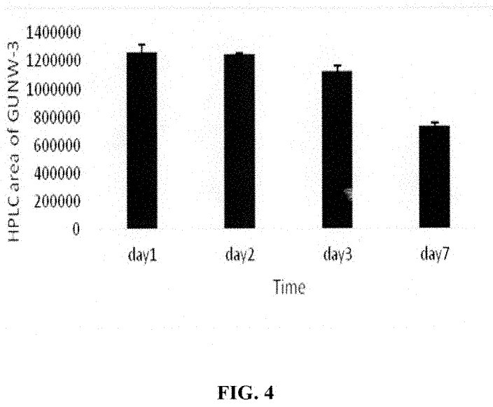

The stability of GUNW-3 in a methanol-ammonium aqueous solution (1:1) at pH 10.8, the pH GUNW-3 was soluble, was checked continuously for 7 days at 4° C. (FIG. 4). GUNW-3 was stable in the first 3 days. However, it started to decompose quickly after 3 days. The accelerated decomposition after day 3 suggests a possibility of a decomposition product-facilitated decomposition (FIG. 4).

### Example 2. Toxicity Study

i. In-Vitro Toxicity

An in-vitro cytotoxicity study by the MTT assay of GUNW-3 revealed IC₅₀ values of 0.65 mM and 0.47 mM for CV-1 cells (monkey kidney cells) and NCI-H226 cells (human lung cancer cells), respectively. The CMC for GUNW-3 was determined to be 3.9 μM (please refer to the GUNW-3 micelle section), much lower than the IC₅₀ values suggesting that GUNW-3 is relatively safe.

A preliminary study was conducted to exam the in vivo toxicity of GUNW-3 in the forms of GUNW-3 micelles and GUNW-3 liposomes in mice. In the preliminary study, two mice were used: one for GUNW-3 micelles and one for GUNW-3 liposomes. No sign of abnormal activities (food intake, weight change, and behave change) were observed when the mice were given continuously for four days at a daily dose that was 3.7 times higher than the single dose of GUNW-3 micelles or GUNW-3 liposomes used for brain-targeting. After five days, mice were sacrificed and examined by a university pathologist for a pathological examination of various organs. No organ toxicity was observed.

GUNW-3 was found to be able to form micelles independently. GUNW-3 micelles were found to be able to enter the brain and carry compounds to the brain.

### Example 3. Determination of the Critical Micellar Concentration (CMC) of GUNW-3

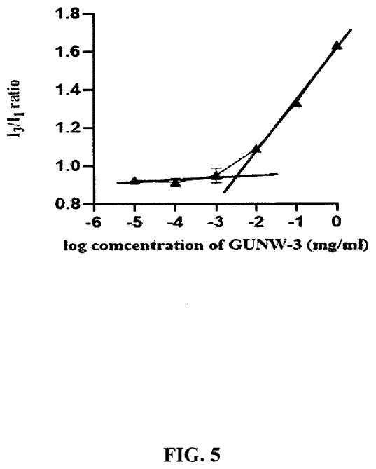

CMC is a critical micelle parameter to determine the stability of micelles and is also a parameter to determine if the micelles are stable enough to be used for a clinical application. The CMC of micelles need to be in μM concentration so that the micelles are stable enough to remain as micelles once being diluted in the blood stream. The CMC of the GUNW-3 micelles was determined to be 3.9 μM by using pyrene—a fluorescens probe. FIG. 5 shows the data from the experiment. Pyrene is a hydrophobic molecule that has a very low water solubility. It showed low but constant fluorescence intensity before GUNW-3 formed micelles (FIG. 5). The fluorescence increased dramatically when GUNW-3 formed micelles—a phenomenon resulted from the fact that pyrene started to be encapsulated inside the micelles which increased substantially the solubility of pyrene. The CMC is determined by the cross point of the two straight lines (FIG. 5). The CMC in low μM of GUNW-3 warrantees a therapeutic application of GUNW-3 micelles.

i. GUNW-3 DiR Micelles

To help determine if GUNW-3 micelles exhibit a brain-targeting effect, DiR, a near infrared fluorescent lipophilic carbocyanine and commonly used dye in determining the in vivo brain targeting ability of nanoparticles, is encapsulated in the GUNW-3 micelles to help track the location of the GUNW-3 micelles in mice. DiR is also a hydrophobic molecule. Therefore, this experiment also helps demonstrate the ability of GUNW-3 micelles to deliver a hydrophobic compound.

ii. Preparation of GUNW-3 DiR Micelles

The GUNW-3 DiR micelles were prepared by a film-dispersion method. Briefly, 70 mg of GUNW-3 and 0.25 mg of DiR were suspended in ethanol (50 μl) and vortex-mixed. The ethanol was evaporated by using nitrogen to form the film. The residue was hydrated with Dulbecco's phosphate buffer saline (DPBS) to a concentration of 7 mg/mL of GUNW-3. The GUNW-3 micelle solution was centrifuged at 14000 rpm for 10 min to remove insoluble DiR. The parameters of GUNW-3 micelles are presented below in Table 1.

### Example 4. Preparation of Control DiR Liposomes

Control DiR liposomes were prepared as one of the two controls for GUNW-3 micelles for brain-targeting experiments in mice. The liposomes are cationic liposomes and known to exhibit some brain-targeting effects. Therefore, cationic liposomes serve as a good positive control. The control DiR liposomes were prepared by the Thin Layer Hydration method. Briefly, lecithin (7 mg/ml), cholesterol (1 mg/ml), dimethyldioctadecyl-ammonium bromide (DDAB) (2 mg/ml), and DiR (0.025 mg/ml) were dissolved in chloroform. The thin film was formed after rotavapory evaporation of solvents overnight under a reduced pressure. The thin film was then hydrated using DPBS solution (1 mL) (usually 5 mL will be used to prepare a batch) for 10 min and vortex-mixed for 2 min. Liposome size reduction was achieved by a bath sonicator for 20 min (4 min sonication with 1 min break) followed by extrusion through a 200 nm, and then 100 nm filter. A Sephadex column (PD 10 column, GE health care, Little Chalfont, UK) was used to separate untrapped DiR by centrifugation at 2500 rpm for 2 min to yield the control DiR liposomes. The parameters of the control DiR liposomes are presented in Table 1.

TABLE 1
Parameters of GUNW-3 micelles and control DiR liposomes.
Results are presented as mean ± SD (n = 3).

| | Theoretical | | Zeta potential | Encapsulation | Loading |
|---|---|---|---|---|---|
| Formulation | loading (%) | Size (nm) | (mV) | efficiency (%) | (%) |
| GUNW-3 DiR micelles | 0.35 | 29.1 ± 5 | −19 ± 2.1 | 68 ± 0.6 | 0.24 |
| Control DiR liposomes | 0.25 | 99.5 ± 2.5 | +19.2 ± 6.5 | 67.7 ± 0.7 | 0.16 |

### Example 5. Stabilities of GUNW-3 DiR Micelles and Control DiR Liposomes

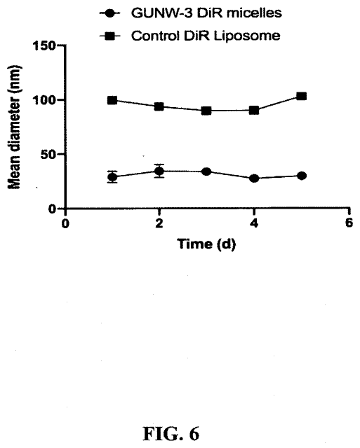

The particle size was used as a parameter for the measurement of the stabilities of GUNW-3 DiR liposomes and control DiR liposomes. FIG. 6 shows that both GUNW-3 DiR micelles and control DiR liposomes were stable over a period of 5 days at 4° C. —the storage temperature (FIG. 6). Data are presented as mean±SD (n=3).

### Example 6. Stabilities of GUNW-3 DiR Micelles and Control DiR Liposomes in the Presence of Fetal Bovine Serum (FBS). Data are Presented as Mean±SD (n=3)

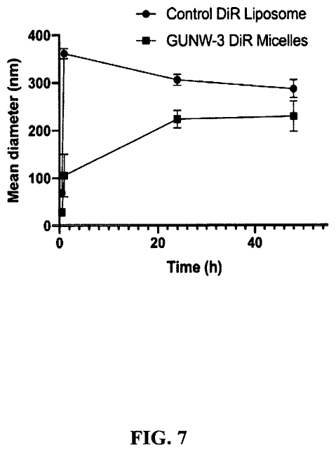

Since micelles will encounter proteins, mainly albumin, once in the blood stream, the stability of GUNW-3 micelles in the presence of FBS was checked at 37° C. As shown in the figure, the particle size of GUNW-3 DiR micelles increased from ˜20 nm to ˜110 nm then continued to increase to reach 210 nm at 24 h. The particle size of GUNW-3 micelles remained as 210 nm after 24 h until 48 h when the experiment ended. The results suggest that the particle size of GUNW-3 DiR micelles was increased probably through complexed with FBS to form a larger particle (210 nm). Similar to GUNW-3 DiR micelles, control DiR liposomes quickly complexed with FBS changing the particle size from ˜100 nm to ˜350 nm. It is noted that the required particle size for brain-targeting should be below ˜150 nm. Larger particles will not be able to enter the brain. Therefore, an increase in particle size observed with GUNW-3 micelles and control DiR liposomes will have impacts on their abilities to enter the brain. (FIG. 7). Data are presented as mean±SD (n=3).

### Example 7. Brain-Targeting of GUNW-3 DiR Micelles

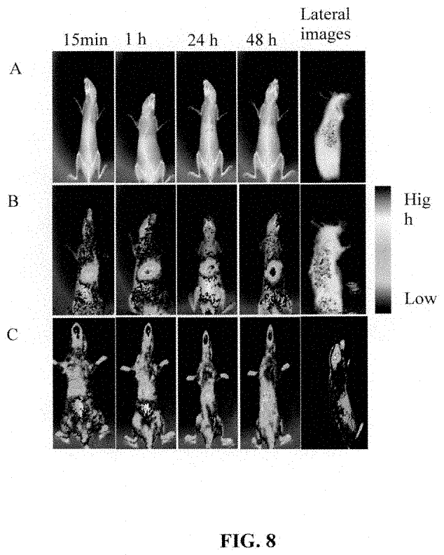

Brain-targeting of GUNW-3 DiR micelles was investigated through whole body imaging on a Bruker Xtream in-vivo imager in mice after a tail vein injection of GUNW-3 DiR micelles. Control DiR liposomes and free DiR dissolved in ethanol (5 μg/25 μL injected) were used as two controls. The results were presented in FIG. 8. As demonstrated in FIG. 8C, GUNW-3 DiR micelles distributed to the brain rapidly within 15 min. A minimum amount of control DiR liposomes were observed in the brain (FIG. 8B) consistent with the knowledge that cationic liposomes have some brain-targeting abilities. No significant fluorescence was observed in the brain of the mice treated with free DiR (FIG. 8A) consistent with the fact that DiR is not able to pass the BBB. Lateral images at 1 h further confirm a significant brain distribution of GUNW-3 DiR micelles, a minimum brain distribution of control DiR liposomes, and no significant brain distribution of free DiR. The lateral image of the mouse treated with GUNW-3 micelles also showed the presence of the micelles in the spinal cord which was not observed for control DiR liposomes and free DiR. The brain fluorescence intensity remained strong in mice treated with GUNW-3 DiR micelles at 48 h confirming a retention of GUNW-3 DiR micelles in the mouse brain after 48 h.

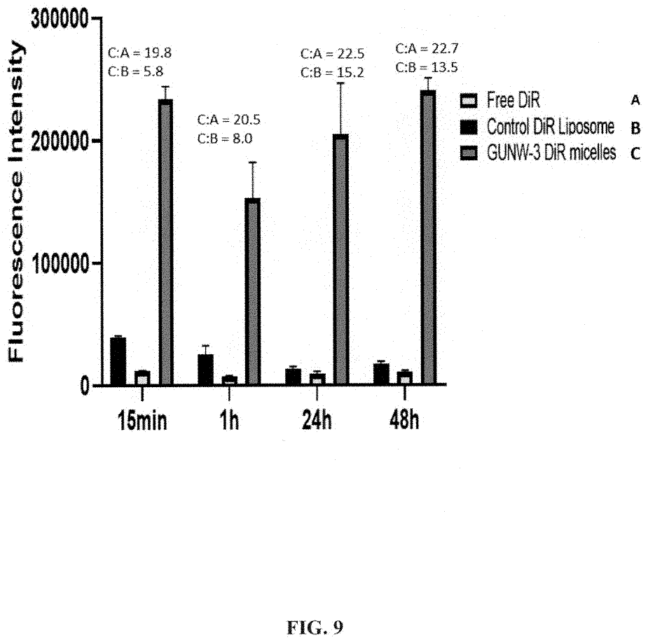

FIG. 9 shows a fold of increase in brain targeting when comparing GUNW-3 DiR micelles with control DiR liposomes (C:B) and with free DiR (C:A) based on the fluorescence intensity in FIG. 8. An about 6 to 15-fold increase in brain targeting was observed for GUNW-3 micelles when compared with the control DiR liposomes (C:B).

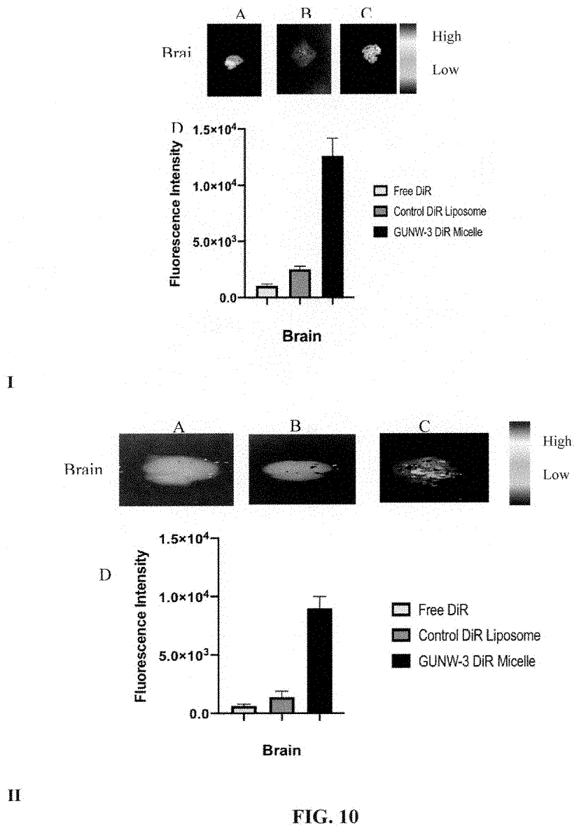

FIG. 10-I and FIG. 10-II were images of isolated brains from mice treated with a treatment for 1 h and 48 h respectively. The brains were collected after heart perfusion with DPBS to remove blood in tissues. The images confirm that GUNW-3 liposomes delivered DiR effectively to the brain while control liposomes and free DiR did not.

### Example 8. The Ability of GUNW-3 Micelles to Deliver Tamoxifen to the Brain

The ability of GUNW micelles to deliver a drug to the brain by using Tamoxifen as a model drug was also tested.

i. Preparation of GUNW-3 Tamoxifen Micelles

A mixture of 3 mg of tamoxifen and GUNW-3 (7 mg/ml) dissolved in THF (15 mL) and deionized water (1 mL) in a 50 mL flask was rotavaped to remove solvents for 20 min at 60° C. to form dried film. The dried film was hydrated with DPBS (10 mL) through the use of a rotavapor for 15 min at 60° C. followed by sonication for 4 min with 1 min break for 4 times at 37° C., A Sephadex column (PD 10 column, GE health care, Little Chalfont, UK) was used to separate untrapped tamoxifen. The column was centrifuged at 2500 rpm for 2 min to remove untrapped tamoxifen. The parameters of GUNW-3 Tamoxifen micelles are presented in Table 2.

TABLE 2
Parameters of GUNW-3 Tamoxifen micelles

| Parameters | GUNW-3 micelles |
|---|---|
| Particle size (nm) | 262.3 |
| Zeta potential (mv) | −37.6 |
| PDI | 0.31 |
| EE % | 52% |
| LC % | 25.8% |

ii. Brain-Targeting of GUNW-3 Tamoxifen

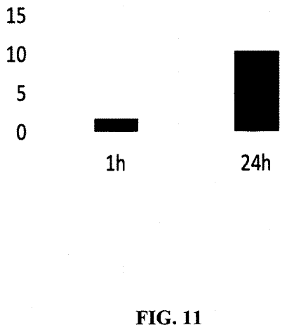

The fold of increase in brain Tamoxifen concentration delivered by GUNW-3 micelles were 1.7 and 10.4 at 1 h and 24 h respectively when compared with Tamoxifen given by IV (n=1)(FIG. 11).

### Example 9. GUNW-3 DiR Liposomes

To help determine if GUNW-3 liposomes exhibit a brain-targeting effect, DiR, a near infrared fluorescent lipophilic carbocyanine and commonly used dye in determining in vivo brain targeting ability of nanoparticles, is encapsulated in the GUNW-3 liposomes to help track the location of GUNW-3 liposomes in mice. DiR is also a hydrophobic molecule. Therefore, this experiment also helps demonstrate the ability of GUNW-3 liposomes to deliver a hydrophobic compound.

i. Preparation of GUNW-3 DiR Liposomes

GUNW-3 DiR liposomes were prepared by using the Thin Layer Hydration technique. Briefly, Lecithin (7 mg/ml), cholesterol (1 mg/ml), DDAB (2 mg/ml), and DiR (0.025 mg/ml) were dissolved in chloroform. To form a thin film, solution was evaporated by rotavapory evaporation overnight under the reduced pressure. The thin film was hydrated with a DPBS solution (1 mL) (usually 5 mL will be used to prepare a batch) containing GUNW-3 (7 mg/mL) for 10 min followed by vortex-mixing for 2 min. To obtain the desired size, a bath sonicator was employed to sonicate the mixture for 20 min (4 min sonication with 1 min break) followed by extrusion with a 200 nm then 100 nm filter. A Sephadex column (PD 10 column, GE health care, Little Chalfont, UK) was used to separate untrapped DiR by centrifugation at 2500 rpm for 2 min to yield the GUNW-3 DiR liposomes.

The liposomes were diluted (0.5:100) with deionized water before used for size and zeta potential determination using the dynamic light scattering (DLS) method on a Zetasizer (Malvern instrument, Westborough, Mass.).

The control DiR liposomes were prepared in the same way except DPBS, instead of DPBS containing GUNW-3, was used to hydrate the thin film.

The parameters of GUNW-3 DiR liposomes and control DiR liposomes s are presented below in Table 3.

TABLE 3
Parameters of GUNW-3 DiR liposomes and control DiR
liposomes (n = 3, mean ± SD).

| | | | Zeta | | | |
|---|---|---|---|---|---|---|
| | Theoretical | | potential | Encapsulation | Drug | |
| Formulation | loading (%) | Size (nm) | (mV) | efficiency (%) | loading (%) | GUNW-3 |
| GUNW-3 DiR | 0.22 | 102 ± 0.6 | −27.4 ± 1.9 | 67.1 ± 0.5 | 0.15 | 4..48 mg/mL |
| liposomes | | | | | | |
| Control DiR | 0.25 | 99.5 ± 2.5 | +19.2 ± 6.5 | 67.7 ± 0.7 | 0.16 | none |
| liposomes | | | | | | |

ii. Stabilities of GUNW-3 DiR Liposomes and Control DiR Liposomes

iiA. Stabilities of GUNW-3 DiR Liposomes and Control DiR Liposomes

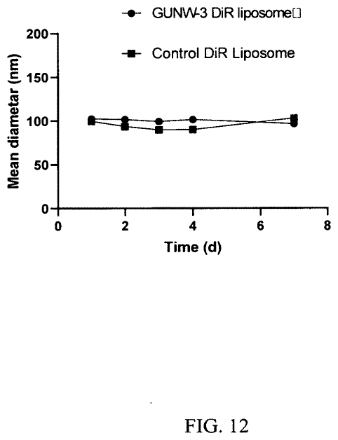

The particle size was used as a parameter to reflect the stability of liposomes. The Stabilities of GUNW-3 DiR liposomes and control DiR liposomes were checked at 4° C. for 7 days. The results are represented in FIG. 12. The sizes of the control DiR liposomes were 99.56±2.89 nm and 102.83±1.2 nm on day 1 and day 7 with no significant statistic difference. Similar results were found with GUNW-3 DiR liposomes. The sizes of the GUNW-3 DiR liposomes were 102±0.6 nm and 96±1.6 nm respectively on day 1 and day 7 with no significant statistic difference. These results suggest that both GUNW-3 DiR liposomes and control DiR liposomes were stable for 7 days at 4° C. —a storage temperature. Results are presented as mean±SD (n=3).

iiB. Stabilities of GUNW-3 DiR Liposomes and Control DiR Liposomes in the Presence of FBS

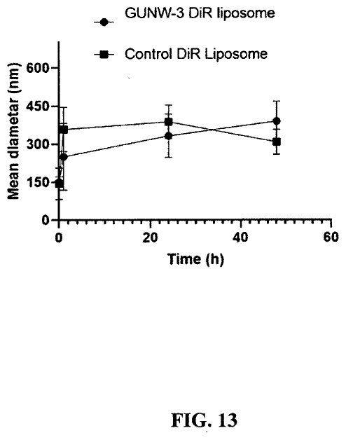

Since liposomes will encounter proteins, once in the blood stream, the stability of GUNW-3 liposomes in the presence of proteins were investigated by using FBS as model proteins at 37° C. As shown in FIG. 13, the particle sizes of GUNW-3 DiR liposomes increased from ˜100 nm to ˜220 nm within 2 h, then slowly and continuously increased to ˜400 nm in 48 h while control DiR liposomes quickly increased from ˜100 nm to ˜360 nm within 2 h and maintained the sizes for the remaining period. These results suggest that control DiR liposomes quickly attached to FBS and increased the particle size while attachment of GUNW-3 DiR liposomes to FBS might involve two different binding sites on FBS with the first quick attachment to one binding site followed by a slow attachment to the second binding site. Another possibility for the two-phase particle size increase is that the second phase might be caused by slow particle aggregation. Since the required particle size for brain-targeting should be below ˜150 nm and larger particles will not be able to enter the brain, an increase in particle size observed with GUNW-3 DiR liposomes and control DiR liposomes will have impacts on their abilities to enter the brain. Results are presented as mean±SD (n=3).

### Example 10. Brain-Targeting of GUNW-3 DiR Liposomes

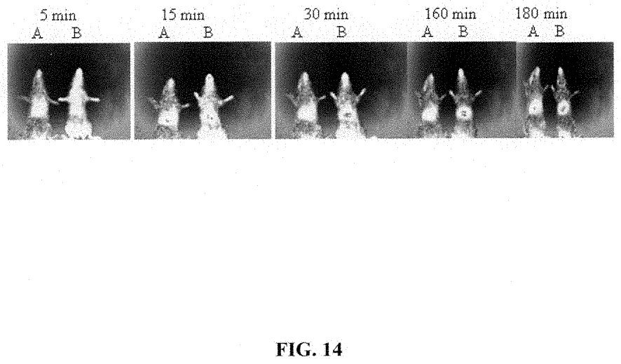

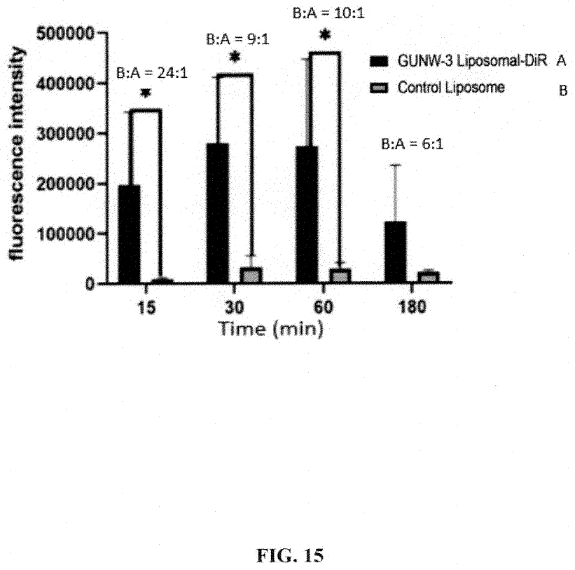

Brain-targeting of GUNW-3 DiR liposomes was investigated through whole body imaging of mice on a Bruker Xtream in-vivo imager after a tail vein injection of GUNW-3 DiR liposomes. Control DiR liposomes were used as a control. The results were presented in FIG. 14. As demonstrated in the figure, GUNW-3 DiR liposomes distributed to the brain rapidly within 5 min (5 min, mouse B). A minimum amount of fluorescence was observed in the brain of the mouse treated with control DiR liposomes (5 min, mouse A) which is consistent with the knowledge that cationic liposomes exhibit some brain targeting effects. The brain fluorescence intensity remained strong in mice treated with GUNW-3 DiR liposomes at 180 min confirming a retention of GUNW-3 DiR liposomes. FIG. 15 provides the fluorescence intensities from the brains at different time points based on FIG. 14. The fold of increase of the brain targeting was presented in Table 4. These results support that GUNW-3 liposomes delivered DiR to the brain more effectively than that by the control DiR liposomes. Results are presented as mean±SEM (n=4).

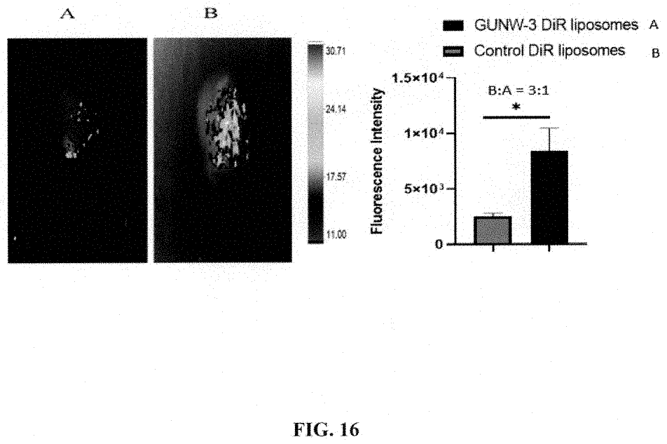

FIG. 16 presents the images of the brains collected after heart perfusion to remove blood in tissues. The fluorescence intensity shows about a 3 fold increase in DiR delivered to the brain by GUNW-3 liposomes when compared with control liposomes (FIG. 16C).

It is noted that the fold of increase in brain targeting obtained based on the fluorescence intensity of the whole body imaging was higher than that obtained based on the fluorescence intensity of the isolated brains.

TABLE 4
Results presented as a ratio of brain fluorescence intensity
from a mouse treated with GUNW-3 DiR liposomes (B) vs.
that from the control DiR liposomes (A) as presented in FIG. 16.

Time (min)

| | 15 | 30 | 60 | 180 |
|---|---|---|---|---|
| B:A (brain | 24 | 9 | 10 | 6 |
| fluorescence ratio) | | | | |

### Example 11. The Ability of GUNW-3 Liposomes to Deliver Tamoxifen to the Brain

The ability of GUNW liposomes to deliver a drug to the brain by using Tamoxifen as a model drug was also investigated.

i. Preparation of GUNW-3 Tamoxifen Liposomes

GUNW-3 Tamoxifen liposomes were prepared by the Thin Layer Hydration method. Briefly, Lecithin (7 mg/ml), cholesterol (1 mg/ml), DDAB (2 mg/ml), and tamoxifen (3 mg/ml) were dissolved in chloroform. The thin film formation was achieved by rotavapory evaporation of solvents overnight under a reduced pressure. The thin film was hydrated by a DPBS solution containing GUNW-3 (7 mg/mL) for 10 min followed by vortex-mixing for 2 min. To obtain the desired size, a bath sonicator was employed to sonicate the mixture for 20 min (4 min sonication with 1 min break) followed by extrusion using a 200 nm then 100 nm filter. A Sephadex column (PD 10 column, GE health care, Little Chalfont, UK) was used to separate untrapped Tamoxifen by centrifugation at 2500 rpm for 2 min to yield the GUNW-3 Tamoxifen liposomes. The liposomes were diluted (0.5:100) with deionized water before used for size and zeta potential determination using the dynamic light scattering (DLS) method on a Zetasizer (Malvern instrument, Westborough, Mass.).

Control Tamoxifen liposomes were prepared in the same manner except no GUNW-3 was included.

The parameters of GUNW-3 Tamoxifen liposomes and control Tamoxifen liposomes are listed in Table 5.

TABLE 5
Nanoparticle parameters of the liposomes

| Nanoparticle | Control tamoxifen | GUNW-3 tamoxifen |
|---|---|---|
| parameters | liposomes | liposomes |
| Particle size (nm) | 117 ± 9 nm | 105 ± 2.5 nm |
| Zeta potential (mv) | +38 ± 7 mV | −31.1 ± 2 mV |
| Tamoxifen EE % | 62% | 89% |
| LC % | 20.3% | 25% |
| Phosphatidylcholine | 7 mg/mL | 7 mg/mL |
| Cholesterol | 1 mg/mL | 1 mg/mL |
| Cationic lipid (DDAB) | 3 mg/mL | 3 mg/mL |
| GUNW-3 | — | 1.2 mg/mL |

ii. Stabilities of GUNW-3 Tamoxifen Liposomes and Control Tamoxifen Liposomes

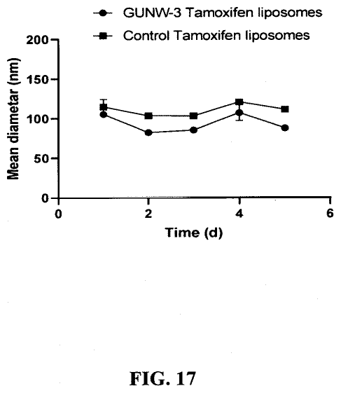

The stabilities of GUNW-3 Tamoxifen liposomes and control Tamoxifen liposomes were determined by following the change in particle size for 5 days at 4° C. —a storage temperature (FIG. 17). The particle sizes of the control tamoxifen liposomes were 114.6±9 nm and 111.3±0.5 nm on day 1 and day 5 respectively, not statistically different. The particle sizes of the GUNW-3 tamoxifen liposomes were 105.3±2 0.5 nm and 87.6±2 nm on day 1 and day 5 respectively which were statistically different. (n=3, p<0.05)

### Example 12. Brain-Targeting of GUNW-3 Tamoxifen Liposomes

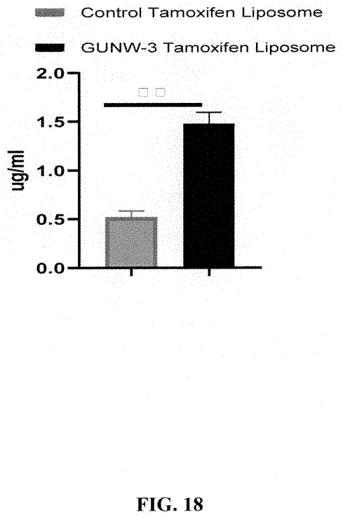

The fold of increase in brain Tamoxifen accumulation delivered by GUNW-3 Tamoxifen liposomes were about 3 fold at 1 h when compared to that delivered by the control Tamoxifen liposomes (n=3, p<0.01)(FIG. 18).

### Example 13. Brain-Targeting of GUNW-3 Micelles after Co-Treatment of GSH

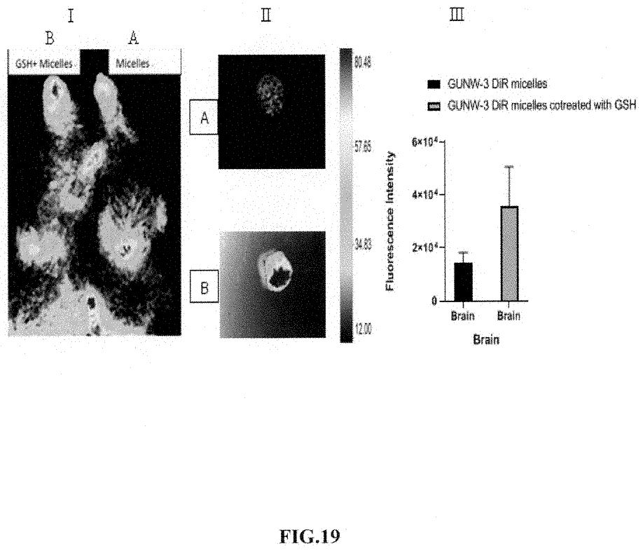

It was found that co-injection of GSH further increased the brain targeting effect of GUNW-3 micelles. Representative in vivo whole body images obtained from mice treated with DiR delivered by GUNW-3 DiR micelles (A) and DiR delivered by GUNW-3 DiR micelles with a co-injection of GSH (B) are presented in FIG. 19-I. FIG. 19-II shows images derived from the corresponding isolated brain after heart perfusion with DPBS to remove blood in tissues. FIG. 19-III is a bar graph demonstrating the fluorescence intensity from the corresponding brain in FIG. 19-II. As shown in FIG. 19-III, a further ˜2 fold increase in brain targeting was observed when GUNW-3 DiR micelles was co-injected with GSH.

### Example 14. GUNW-3 Micelles Increase Taxol's Aqueous Solubility for IV Injection

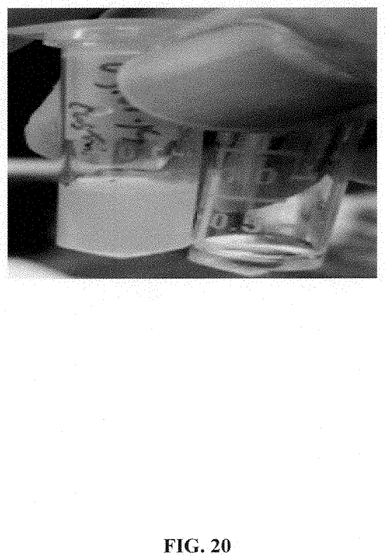

Taxol is a drug used for a number of types of cancer. The major challenge in using taxol for therapeutic treatments is its extremely poor water solubility. In our investigation of GUNW-3, we found GUNW-3 can help Taxol dissolve in water. FIG. 20 show two vials with one vial containing a clinically used IV injection solution which was initially a clear solution but became cloudy later while the other vial containing the same amount of taxol but remained as a clear solution when GUNW-3 was used. The results suggest that GUNW-3 might be able to help Taxol form a stable IV injection solution which will be useful in clinics. The results also suggest GUNW-3 might be able to help dissolve other drugs or compounds in addressing their poor water solubility issue for clinical application.

Although the invention has been described with reference to the above examples, it will be understood that modifications and variations are encompassed within the spirit and scope of the invention. Accordingly, the invention is limited only by the following claims.

We claim herein:

1. A blood brain barrier permeable composition, wherein the composition comprises a molecule as set forth in Formula (I), (II), (III), (IV), (V), (VI), (VII), or (VIII): 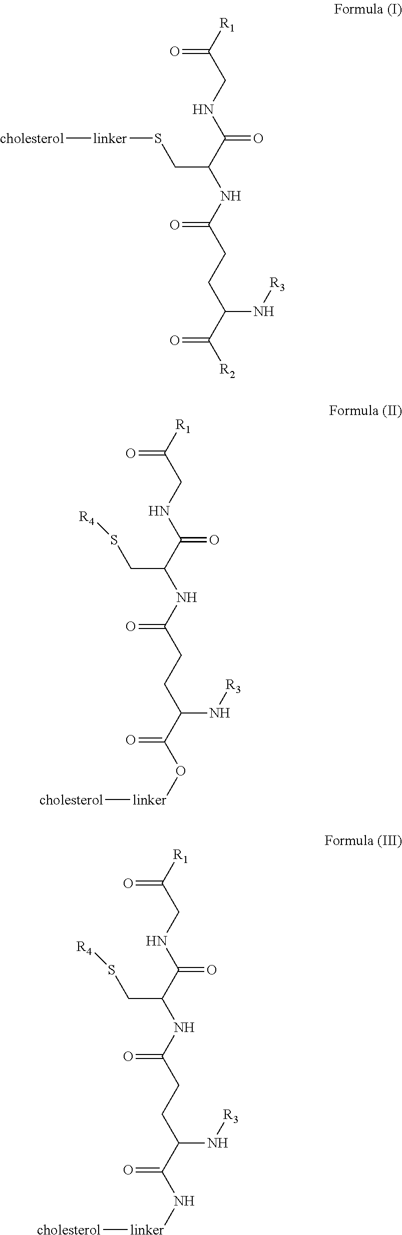 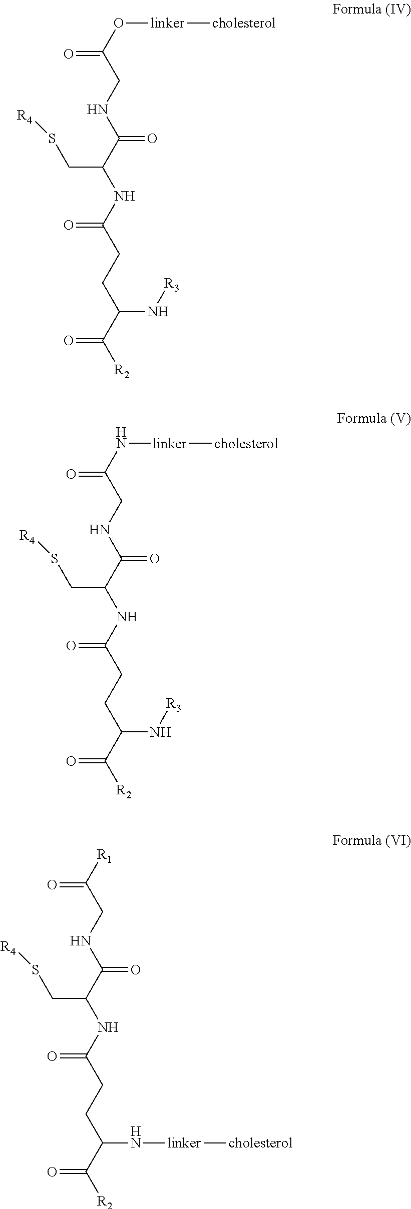 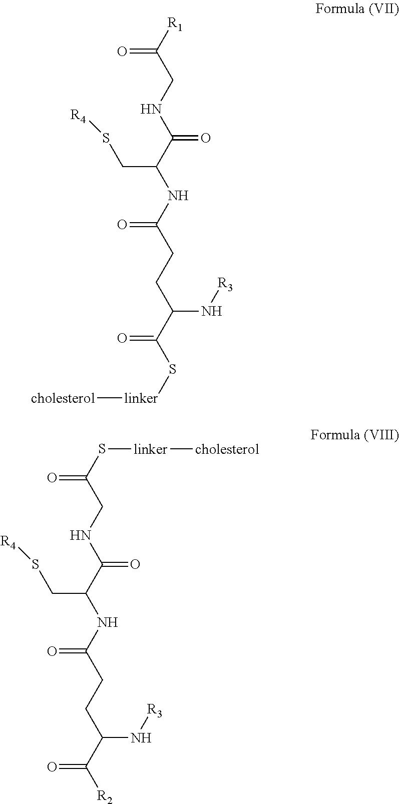
wherein R₁=OH, O-alkyl (1 to 30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), combinations thereof;
R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
R₃=H, alkyl (1-30 C), aryl (6-30 C), or aryl and alkyl combinations (6-30 C);
R₄=H, alkyl (1-30 C), aryl (6-30 C), or aryl and alkyl combinations (6-30 C), and
the linker includes alkyl, aliphatic chain, alkoxy and combinations thereof, wherein the composition does not fluoresce in the absence of an exogenous fluorescing compound.

2. The blood brain barrier permeable composition of claim 1, wherein the composition comprises a molecule as set forth in Formula (I).

3. The blood brain barrier permeable composition of claim 2, wherein the composition comprises a molecule as set forth in Formula (IX):

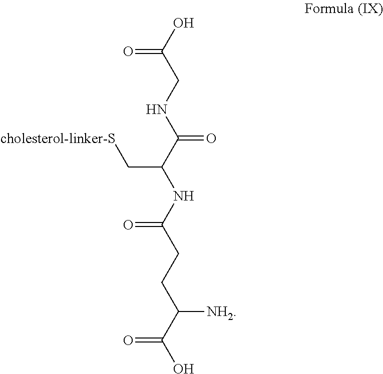

4. The blood brain barrier permeable composition of claim 1, wherein said linker comprises at least one ethylene glycol moiety.

5. The blood brain barrier permeable composition of claim 4, wherein said linker comprises two ethylene glycol moieties.

6. The blood brain barrier permeable composition of claim 1, wherein said composition exhibits a critical micellular concentration (CMC) of about 3.9 μM.

7. A blood brain barrier permeable composition, wherein the composition comprises a molecule as set forth in formula (I), (II), (III), (IV), (V), (VI), (VII), or (VIII): 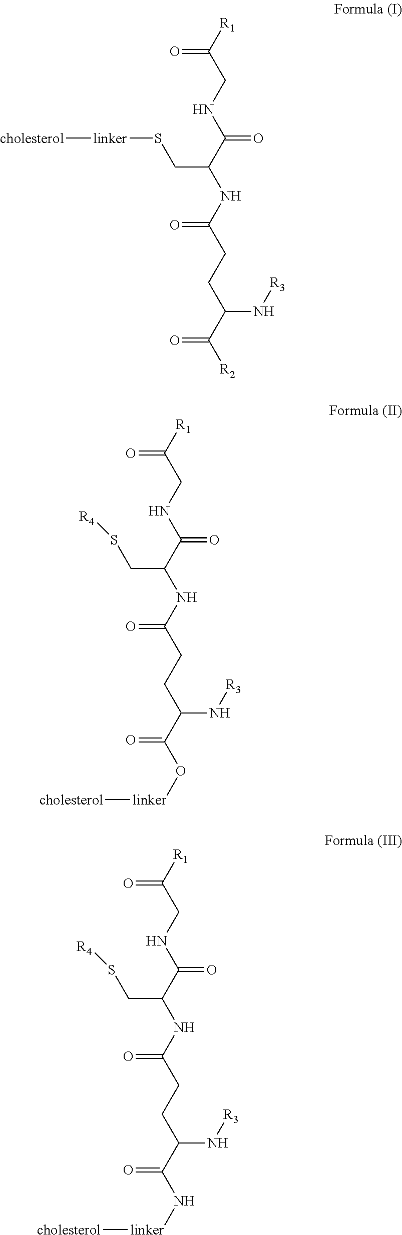 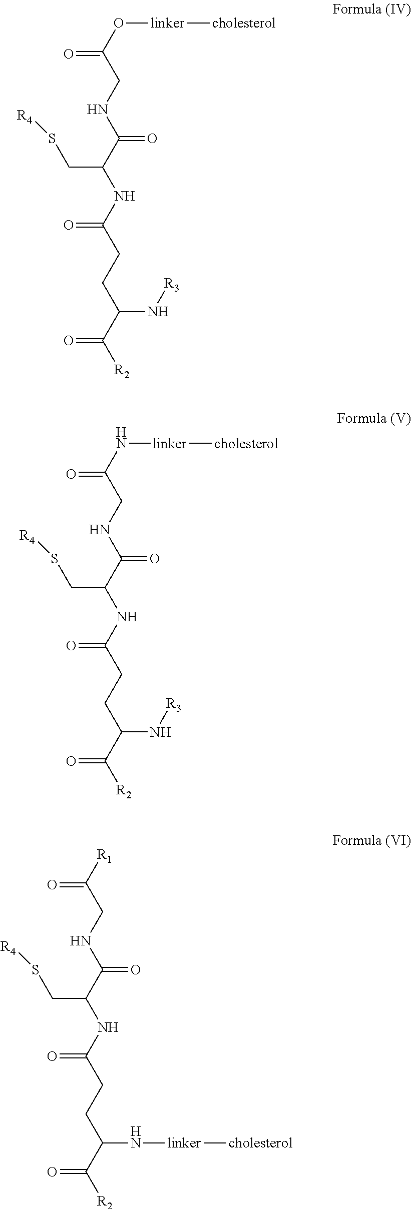 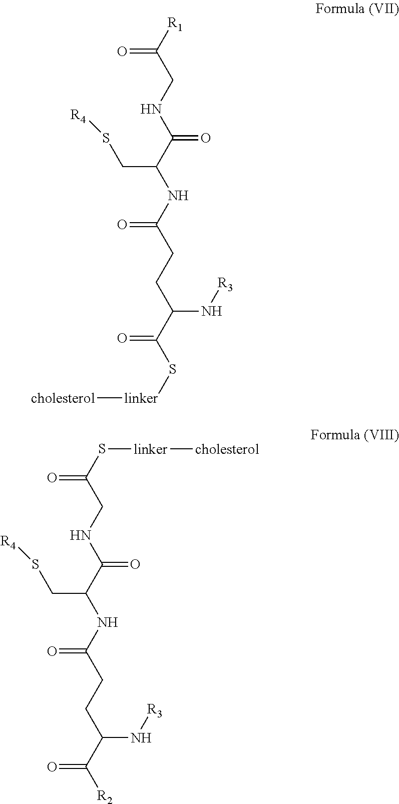
wherein R₁=R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
wherein R₃=R₄=H, alkyl (1-30 C), aryl (6-30 C), or aryl and alkyl combinations (6-30 C), and
the linker includes alkyl, aliphatic chain, alkoxy and combinations thereof, wherein the composition forms micelles.

8. The blood brain barrier permeable composition of claim 7, wherein said linker comprises at least one ethylene glycol moiety.

9. The blood brain barrier permeable composition of claim 4, wherein said linker comprises two ethylene glycol moieties.

10. The blood brain barrier permeable composition of claim 1 or claim 7, wherein the composition further comprises a one or more of the agents selected from the group consisting of buffers, liposomes, dyes, fluorescent agents, drugs, chemotherapeutics, nutraceuticals, vitamins, nucleic acids, proteins, antigens, antibodies buffer, a hydrophobic agent, a therapeutic agent, an antioxidant, an anti-inflammatory drug, an anticancer drug, a cosmetic, a pharmaceutical, a pesticide, an insecticide, an herbicide, an antiseptic, a diagnostic agent, a food additive, a fragrance, and a textile and combinations thereof.

11. The blood brain barrier permeable composition of claim 3, further comprising a hydrophobic agent.

12. The blood brain barrier permeable composition of claim 11, wherein the hydrophobic agent is selected from the group consisting of abietic acid, aceglatone, acenaphthene, acenocoumarol, acetohexamide, acetomeroctol, acetoxolone, acetyldigitoxins, acetylene dibromide, acetylene dichloride, acetylsalicylic acid, alantolactone, aldrin, alexitol sodium, allethrin, allylestrenol, allyl sulfide, alprazolam, aluminum bis(acetylsalicylate), ambucetamide, aminochlothenoxazin, aminoglutethimide, amyl chloride, androstenediol, anethole trithone, anilazine, anthralin, Antimycin A, aplasmomycin, arsenoacetic acid, asiaticoside, astemizole, aurodox, aurothioglycanide, 8-azaguanine, azobenzene; baicalein, Balsam Peru, Balsam Tolu, barban, baxtrobin, bendazac, bendazol, bendroflumethiazide, benomyl, benzathine, benzestrol, benzodepa, benzoxiquinone, benzphetamine, benzthiazide, benzyl benzoate, benzyl cinnamate, bibrocathol, bifenox, binapacryl, bioresmethrin, bisabolol, bisacodyl, bis(chlorophenoxy)methane, bismuth iodosubgallate, bismuth subgallate, bismuth tannate, Bisphenol A, bithionol, bornyl, bromoisovalerate, bornyl chloride, bornyl isovalerate, bornyl salicylate, brodifacoum, bromethalin, broxyquinoline, bufexamac, butamirate, butethal, buthiobate, butylated hydroxyanisole, butylated hydroxytoluene; calcium iodostearate, calcium saccharate, calcium stearate, capobenic acid, captan, carbamazepine, carbocloral, carbophenothin, carboquone, carotene, carvacrol, cephaeline, cephalin, chaulmoogric acid, chenodiol, chitin, chlordane, chlorfenac, chlorfenethol, chlorothalonil, chlorotrianisene, chlorprothixene, chlorquinaldol, chromonar, cilostazol, cinchonidine, citral, clinofibrate, clofazimine, clofibrate, cloflucarban, clonitrate, clopidol, clorindione, cloxazolam, coroxon, corticosterone, coumachlor, coumaphos, coumithoate cresyl acetate, crimidine, crufomate, cuprobam, cyamemazine, cyclandelate, cyclarbamate cymarin, cypernethril; dapsone, defosfamide, deltamethrin, deoxycorticocosterone acetate, desoximetasone, dextromoramide, diacetazoto, dialifor, diathymosulfone, decapthon, dichlofluani, dichlorophen, dichlorphenamide, dicofol, dicryl, dicumarol, dienestrol, diethylstilbestrol, difenamizole, dihydrocodeinone enol acetate, dihydroergotamine, dihydromorphine, dihydrotachysterol, dimestrol, dimethisterone, dioxathion, diphenane, N-(1,2-diphenylethyl)nicotinamide, dipyrocetyl, disulfamide, dithianone, doxenitoin, drazoxolon, durapatite, edifenphos, emodin, enfenamic acid, erbon, ergocominine, erythrityl tetranitrate, erythromycin stearate, estriol, ethaverine, ethisterone, ethyl biscoumacetate, ethylhydrocupreine, ethyl menthane carboxamide, eugenol, euprocin, exalamide; febarbamate, fenalamide, fenbendazole, fenipentol, fenitrothion, fenofibrate, fenquizone, fenthion, feprazone, flilpin, filixic acid, floctafenine, fluanisone, flumequine, fluocortin butyl, fluoxymesterone, fluorothyl, flutazolamn, fumagillin, 5-furfuryl-5-isopropylbarbituric acid, fusafungine, glafenine, glucagon, glutethimide, glybuthiazole, griseofulvin, guaiacol carbonate, guaiacol phosphate, halcinonide, hematoprophyrin, hexachlorophene, hexestrol, hexetidine, hexobarbital, hydrochlorothiazide, hydrocodone, ibuproxam, idebenone, indomethacin, inositol niacinate, iobenzamic acid, iocetamic acid, iodipamide, iomeglamic acid, ipodate, isometheptene, isonoxin, 2-isovalerylindane-1,3-dione, josamycin, 11-ketoprogesterone, laurocapram, 3-0-lauroylpyridoxol diacetate, lidocaine, lindane, linolenic acid, liothyronine, lucensomycin, mancozeb, mandelic acid, isoamyl ester, mazindol, mebendazole, mebhydroline, mebiquine, melarsoprol, melphalan, menadione, menthyl valerate, mephenoxalone, mephentermine, mephenyloin, meprylcaine, mestanolone, mestranol, mesulfen, metergoline, methallatal, methandriol, methaqualone, 3-methylcholanthrene, methylphenidate, 17-methyltestosterone, metipranolol, minaprine, myoral, naftalofos, naftopidil, naphthalene, 2-naphthyllactate, 2-(2-naphthyloxy)ethanol, naphthyl salicylate, naproxen, nealbarbital, nemadectin, niclosamide, nicoclonate, nicomorphine, nifuroquine, nifuroxazide, nitracrine, nitromersol, nogalamycin, nordazepamn, norethandrolone, norgestrienone; octaverine, oleandrin, oleic acid, oxazepam, oxazolam, oxeladin, oxwthazaine, oxycodone, oxymesterone, oxyphenistan acetate, paclitaxel, paraherquamide, parathion, pemoline, pentaerythritol tetranitrate, pentylphenol, perphenazine, phencarb amide, pheniramine, 2-phenyl 6-chlorophenol, phentlmethylbarbituric acid, phenyloin, phosalone, phthalylsulfathiazole, phylloquinone, picadex, pifamine, piketopfen, piprozolin, pirozadil, plafibride, plaunotol, polaprezinc, polythiazide, probenecid, progesterone, promegestone, propanidid, propargite, propham, proquazone, protionamide, pyrimethamine, pyrimithate, pyrvinium pamoate, quercetin, quinbolone, quizalofo-ethyl, rafoxanide, rescinnamine, rociverine, runnel, salen, scarlet red, siccanin, simazine, simetride, sobuzoxane, solan, spironolactone, squalene, stanolone, sucralfate, sulfabenz, sulfaguanole, sulfasalazine, sulfoxide, sulpiride, suxibuzone, talbutal, tamoxifen, terguide, testosterone, tetrabromocresol, tetrandrine, thiacetazone, thiocolchicine, thioctic acid, thioquinox, thioridazine, thiram, thymyl N isoamylcarbamate, tioxidazole, tioxolone, tocopherol, tolciclate, tolnaftate, triclosan, triflusal, triparanol, ursolic acid, valinomycin, veraparnil, vinblastine, vitamin A, vitamin D, vitamin E, xenbucin, xylazine, zaltoprofen, zearalenone, and combinations thereof.

13. The blood brain barrier permeable composition of claim 3, wherein said composition is a micelle or a liposome.

14. A method of treating a disorder that affects the brain comprising administering to a subject in need thereof a pharmaceutical composition comprising the blood brain barrier permeable composition of claim 7 at a sufficient concentration to relieve the symptoms of said disorder.

15. The method of claim 14, wherein the disorder is selected from the group consisting of Alzheimer's disease, dementias, brain cancer, epilepsy, seizure disorders, mental disorders, Parkinson's disease, movement disorders, stroke and transient ischemic attacks.

16. The method of claim 14, further comprising co-administration of glutathione.

17. A method of treating a disorder that affects the brain comprising administering to a subject in need thereof a pharmaceutical composition comprising the blood brain barrier permeable composition of claim 1 a sufficient concentration to relieve the symptoms of said disorder.

18. The method of claim 17, wherein the disorder is selected from the group consisting of Alzheimer's disease, dementias, brain cancer, epilepsy, seizure disorders, mental disorders Parkinson's disease, movement disorders, stroke and transient ischemic attacks.

19. A kit comprising:
a) a blood brain barrier permeable composition, wherein the composition comprises a molecule as set forth in:
(i) formula (I), (II), (III), (IV), (V), (VI), (VII), or (VIII): 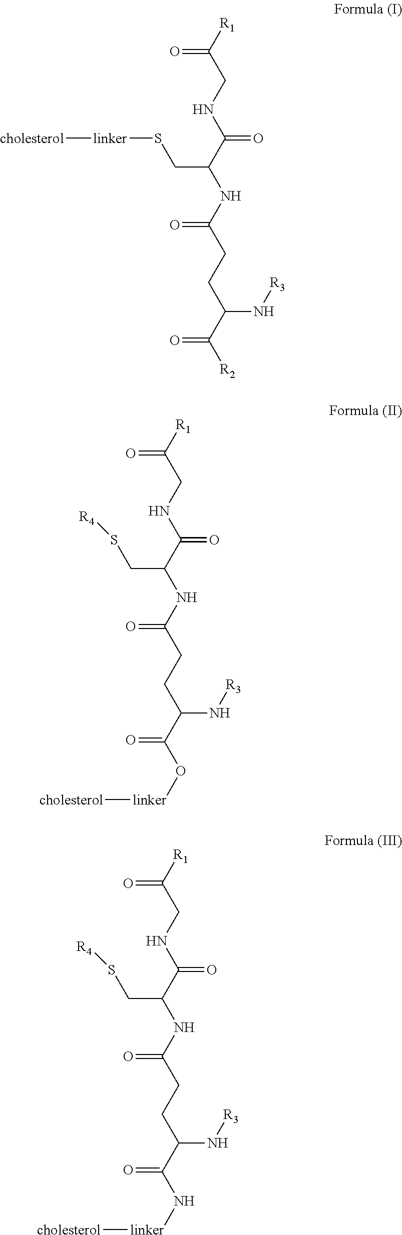 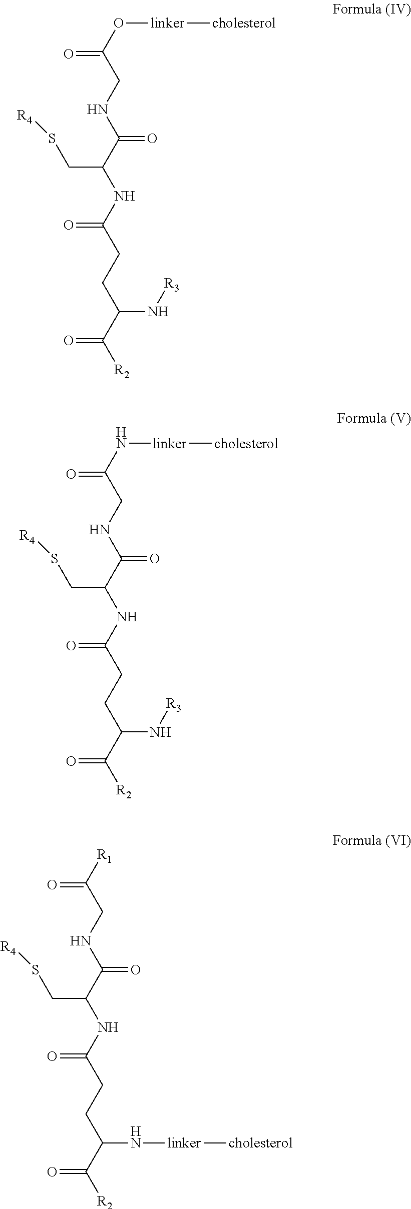 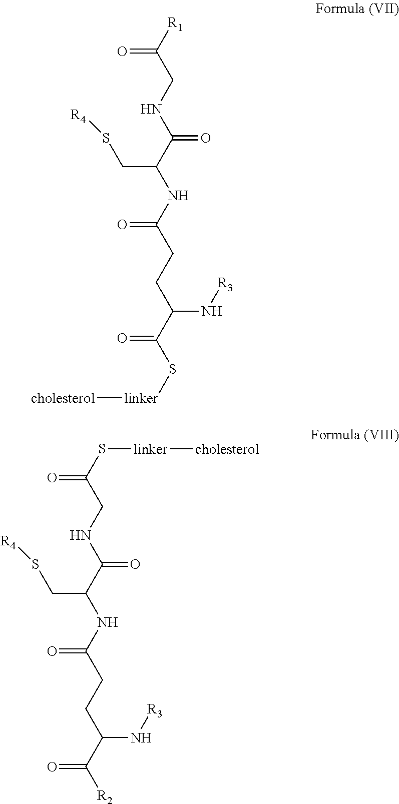
wherein R₁=OH, O-alkyl (1 to 30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), combinations thereof;
R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
R₃=H, alkyl (1-30 C), aryl (6-30 C), or aryl and alkyl combinations (6-30 C),
R₄=H, alkyl (1-30 C), aryl (6-30 C), or aryl and alkyl combinations (6-30 C), and
the linker includes alkyl, aliphatic chain, alkoxy and combinations thereof, wherein the composition does not fluoresce in the absence of an exogenous fluorescing compound or
(ii) a blood brain barrier permeable composition, wherein the composition comprises a molecule as set forth in formula (I), (II), (III), (IV), (V), (VI), (VII), or (VIII): 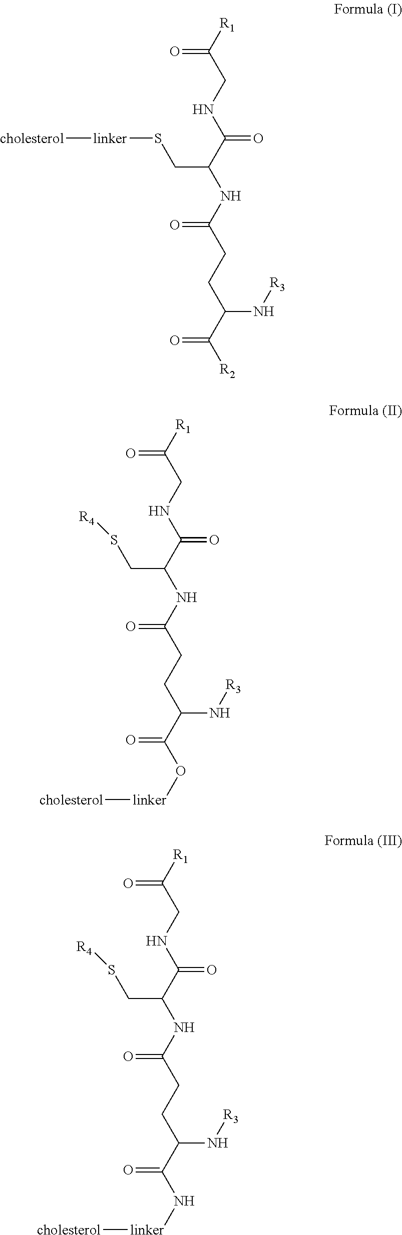 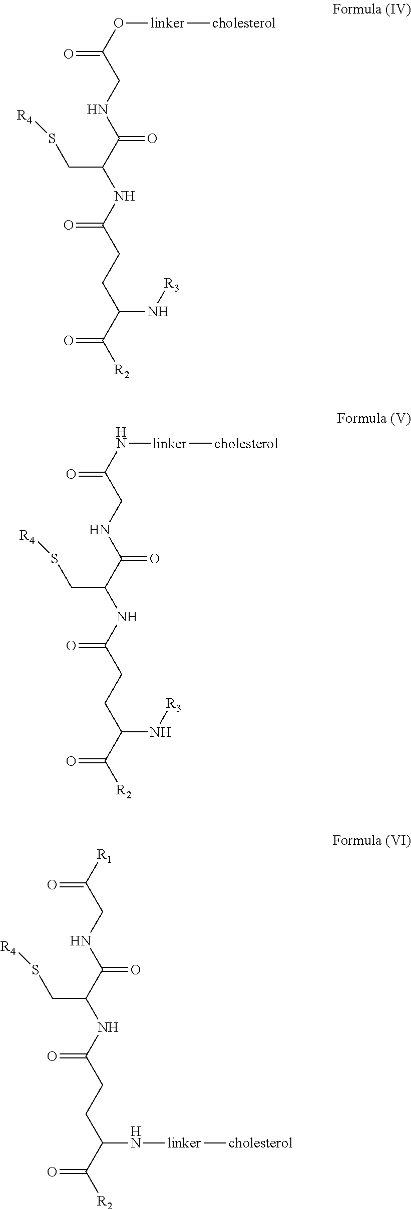 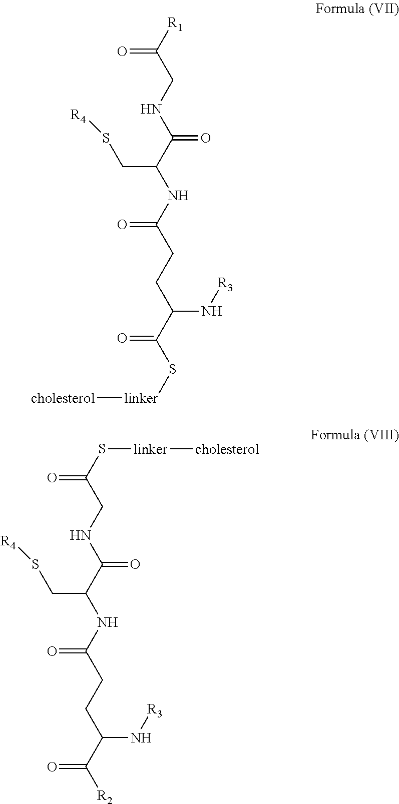
wherein R₁=R₂=OH, O-alkyl (1-30 C), O-aryl (6-30 C), O-aryl and O-alkyl combinations (6-30 C), S-alkyl (1-30 C), S-aryl (6-30 C), S-aryl and S-alkyl combinations (6-30 C), N-alkyl (1-30 C), N-aryl (6-30 C), or N-aryl and N-alkyl combinations (6-30 C), and combinations thereof;
wherein R₃=R₄=H, alkyl (1-30 C), aryl (6-30 C), or aryl and alkyl combinations (6-30 C), and
the linker includes alkyl, aliphatic chain, alkoxy and combinations thereof, wherein the composition forms micelles;
b) optionally, one or more compounds selected from phospholipids, cholesterol or buffers;
c) one or more agents selected from the group consisting of buffers, dyes, fluorescent agents, drugs, chemotherapeutics, nutraceuticals, vitamins, nucleic acids, proteins, antigens, antibodies buffer, a hydrophobic agent, a therapeutic agent, an antioxidant, an anti-inflammatory drug, an anticancer drug, a cosmetic, a pharmaceutical, a pesticide, an insecticide, an herbicide, an antiseptic, a diagnostic agent, a food additive, a fragrance, and a textile and combinations thereof;
d) instructions reciting a method for producing a composition comprising a liposome or micelle and said one or more agents;
e) a label; and
f) a container comprising components (a)-(e).

20. The kit of claim 19, wherein the blood brain barrier permeable composition comprises a molecule as set forth in Formula (IX): [chemical structure]
